(19) United States
(12) Patent Application Publication
(10) Pub. No.: US 2010/0209968 A1
(43) Pub. Date: Aug. 19, 2010

(54) IMMOBILIZED ENZYMES AND USES THEREOF

(75) Inventors: Niki L. Akers, St. Louis, MO (US); James Stirling McLaren, St. Louis, MO (US); Shelley D. Minteer, Pacific, MO (US); Wayne Gellett, Ballwin, MO (US); Mehmet Kesmez, College Station, TX (US); Richard T. Zvosec, Edina, MN (US)

Correspondence Address: SENNIGER POWERS LLP, 100 NORTH BROADWAY, 17TH FLOOR, ST LOUIS, MO, 63102, US

(73) Assignee: AKERMIN, INC., St. Louis, MO (US)

(21) Appl. No.: 12/598,888

(22) PCT Filed: May 5, 2008
(86) PCT No.: PCT/US08/62658

§ 371 Date: Apr. 21, 2010

Related U.S. Application Data
(60) Provisional application No. 60/916,274, filed on May 4, 2007.
(60) Provisional application No. 61/048,202, filed on Apr. 27, 2008.

(51) Int. Cl. C12P 21/06; A23L 1/00; C12P 1/00; C12P 19/00; C12P 17/12; C12P 7/40; C12N 11/00; C02F 3/34

(52) U.S. Cl. 435/68.1; 426/61; 435/41; 435/72; 435/122; 435/136; 435/174; 435/262

(57) ABSTRACT

The present invention generally relates to uses of immobilized enzymes. Immobilized enzymes can be used for various chemical transformations, separations, and purifications and can be used in sensors and diagnostics

### FIELD OF THE INVENTION

The present invention generally relates to uses of immobilized enzymes for various chemical transformations and analyses.

### BACKGROUND OF THE INVENTION

In applied, more particularly in biotechnology, it is known that enzymes, enzyme-producing microorganisms, cells, or cell components can be fixed to certain carriers, particularly if they are used as biocatalysts. This process is known generally as immobilization. Since native enzymes are reduced in their activity by biological, chemical or physical effects during storage or in applications, there is a need to stabilize the enzymes in view of their high production costs. Through immobilization, the enzymes can be reused. After use, the enzymes are easy to remove from the reaction mixture. In this way, they can be used under a variety of processing conditions. Desirably, the substrate and reaction specificity and enzyme reactivity should not be lost as a result of immobilization.

Immobilized enzymes are used in particular in commercially-important biotechnological processes. In the food industry, the use of glucose isomerase to convert glucose to fructose is important. The use of lipase for transesterification of edible oils is another standard process. In other industrial processes, immobilized enzymes are applied in the production of amino acids and in the splitting of penicillin G into 6-aminopenicillic acid.

Immobilized enzyme and cell systems are used not only in biotechnological production processes, but also in analysis, for example in so-called biosensors. The principle of analysis using immobilized systems is based on the reaction of a substrate to be determined by an immobilized enzyme, the changes in the concentrations of product, substrate and co-substrate being able to be followed, for example by several coupled methods (for example enzyme electrodes).

However, current enzyme immobilization materials used to immobilize enzymes for these uses do not significantly stabilize the enzyme to denaturation. Thus, an enzyme immobilization material that increases the stability of enzymes for the disclosed uses is needed.

### SUMMARY OF THE INVENTION

Among the various aspects of the invention is an enzyme immobilized in a micellar or inverted micellar immobilization material capable of immobilizing and stabilizing the enzyme, the material being permeable to a compound smaller than the enzyme. When exposed to a temperature of at least about 65° C. for at least about 18 hours, the stabilized enzyme retains at least about 12% of an activity of an otherwise identical free enzyme exposed to room temperature for the same amount of time. In various embodiments, the stabilized enzyme retains at least 11% or at least about 12, 13, 14, 15, 16, 17, 18, 19, 20, 25, 30, 35, 40, 45, 50, 55, 60, 65, 70, or 75% or more of an activity of an otherwise identical free enzyme exposed to room temperature for the same amount of time.

Another aspect is an enzyme immobilized in a non-naturally occurring or acellular colloidal immobilization material capable of immobilizing and stabilizing the enzyme, the material being permeable to a compound smaller than the enzyme. When exposed to a temperature of at least about 65° C. for at least about 18 hours, the stabilized enzyme retains at least about 12% of an activity of an otherwise identical free enzyme exposed to room temperature for the same amount of time. In various embodiments, the stabilized enzyme retains at least 11% or at least about 12, 13, 14, 15, 16, 17, 18, 19, 20, 25, 30, 35, 40, 45, 50, 55, 60, 65, 70, or 75% or more of an activity of an otherwise identical free enzyme exposed to room temperature for the same amount of time.

Yet another aspect is an enzyme immobilized in a micellar or inverted micellar immobilization material capable of immobilizing and stabilizing the enzyme, the material being permeable to a compound smaller than the enzyme. When exposed to a temperature of at least about 95° C. for at least about 18 hours, the stabilized enzyme retains at least about 1% of an activity of an otherwise identical free enzyme exposed to room temperature for the same amount of time. In various embodiments, the stabilized enzyme retains at least 0.8 or 0.9% or at least about 1, 1.1, 1.2, 1.3, 1.4, 1.5, 2, 3, 4, 5, 6, 7, 8, 9 or 10% or more of an activity of an otherwise identical free enzyme exposed to room temperature for the same amount of time.

A further aspect of the invention is an enzyme immobilized in a non-naturally occurring or acellular colloidal immobilization material capable of immobilizing and stabilizing the enzyme, the material being permeable to a compound smaller than the enzyme. When exposed to a temperature of at least about 95° C. for at least about 18 hours, the stabilized enzyme retains at least about 1% of an activity of an otherwise identical free enzyme exposed to room temperature for the same amount of time. In various embodiments, the stabilized enzyme retains at least 0.8 or 0.9% or at least about 1, 1.1, 1.2, 1.3, 1.4, 1.5, 2, 3, 4, 5, 6, 7, 8, 9 or 10% or more of an activity of an otherwise identical free enzyme exposed to room temperature for the same amount of time.

Another aspect is an enzyme immobilized in a micellar or inverted micellar immobilization material capable of immobilizing and stabilizing the enzyme, the material being permeable to a compound smaller than the enzyme. When exposed to a pH of greater than about 12.5 for at least about 18 hours, the stabilized enzyme retains at least about 9% of an activity of an otherwise identical free enzyme exposed to its optimal pH for the same amount of time. In various embodiments, the stabilized enzyme retains at least 7.5 or 8% or at least about 9, 10, 11, 12, 13, 14, 15, 20, 25, 30, 35, 40, 45, 50, 55, 60, 65, 70, or 75% or more of an activity of an otherwise identical free enzyme exposed to its optimal pH for the same amount of time.

A further aspect is an enzyme immobilized in a non-naturally occurring or acellular colloidal immobilization material capable of immobilizing and stabilizing the enzyme, the material being permeable to a compound smaller than the enzyme. When exposed to a pH of greater than about 12.5 for at least about 18 hours, the stabilized enzyme retains at least about 9% of an activity of an otherwise identical free enzyme exposed to its optimal pH for the same amount of time. In various embodiments, the stabilized enzyme retains at least 7.5 or 8% or at least about 9, 10, 11, 12, 13, 14, 15, 20, 25, 30, 35, 40, 45, 50, 55, 60, 65, 70, or 75% or more of an activity of an otherwise identical free enzyme exposed to its optimal pH for the same amount of time.

Another aspect is an enzyme immobilized in a micellar or inverted micellar immobilization material capable of immobilizing and stabilizing the enzyme, the material being permeable to a compound smaller than the enzyme. When exposed to a pH of less than about 2, the stabilized enzyme retains at least about 10% of its initial bioelectrocatalytic activity for at least about 1 hour when continuously catalyzing a chemical transformation. In various embodiments, the stabilized enzyme retains at least about 10% of its initial bioelectrocatalytic activity for at least about 1, 2, 3, 4, 8, 12, 16, 20, 24, 36, 48, 60, 72, 96, 120, 144, 168, 192, 216 or 240 hours, or more when continuously catalyzing a chemical transformation. In these embodiments, the stabilized enzyme retains at least 9% or at least about 10, 15, 20, 25, 30, 35, 40, 45, 50, 55, 60, 65, 70, 75, 80, 85, 90 or 95% of its initial bioelectrocatalytic activity over that period of time when continuously catalyzing the chemical transformation.

A further aspect is an enzyme immobilized in a non-naturally occurring or acellular colloidal immobilization material capable of immobilizing and stabilizing the enzyme, the material being permeable to a compound smaller than the enzyme. When exposed to a pH of less than about 2, the stabilized enzyme retains at least about 10% of its initial bioelectrocatalytic activity for at least about 1 hour when continuously catalyzing a chemical transformation. In various embodiments, the stabilized enzyme retains at least about 75% of its initial bioelectrocatalytic activity for at least about 1, 2, 3, 4, 8, 12, 16, 20, 24, 36, 48, 60, 72, 96, 120, 144, 168, 192, 216 or 240 hours, or more when continuously catalyzing a chemical transformation. In these embodiments, the stabilized enzyme retains at least 9% or at least about 10, 15, 20, 25, 30, 35, 40, 45, 50, 55, 60, 65, 70, 75, 80, 85, 90 or 95% of its initial bioelectrocatalytic activity over that period of time when continuously catalyzing the chemical transformation.

Among the various aspects of the invention are uses of an immobilized enzyme for catalyzing a reaction wherein the enzyme is immobilized in an enzyme immobilization material. The enzyme immobilization material immobilizes and stabilizes the enzyme, is permeable to a compound smaller than the enzyme, and is a micellar hydrophobically modified polysaccharide.

Other aspects are uses of an immobilized enzyme for detecting an analyte wherein the enzyme is immobilized in an enzyme immobilization material. The enzyme immobilization material immobilizes and stabilizes the enzyme, is permeable to a compound smaller than the enzyme, and is a micellar hydrophobically modified polysaccharide.

Yet other aspects are uses of an immobilized enzyme for catalyzing a reaction wherein the enzyme is immobilized in an enzyme immobilization material. The enzyme immobilization material immobilizes and stabilizes the enzyme and is permeable to a compound smaller than the enzyme. The reaction catalyzed is selected from (a) conversion of carbon dioxide to carbonic acid; (b) enantioselective oxidation or reduction of organic substrates with optional cofactor regeneration; (c) esterification of a carboxylic acid with an alcohol; (d) liquefaction of corn or other cereals; (e) saccharification of corn or other cereals to convert starch into sugars; (f) isomerization of glucose to fructose; (g) synthesis of chiral compounds; (h) interesterification of oils; (i) degumming oil; (j) treating wastewater (reaction); (k) clarifying fruit juice; (l) producing glucose by the starch process; (m) producing glucose and galactose from lactose; (n) synthesizing compounds having peptide bonds; (O) producing 6-aminopenicillic acid from penicillin G; (p) converting sugars to alcohol; (q) removing sulfur from petroleum fractions; (r) converting acrylonitrile to acrylamide; (s) converting 3-cyanopyridine to nicotinamide; (t) reactive separation of a component or components from a mixture and (u) degrading stains in a laundry soil.

A further aspect is an improvement in an enzyme-catalyzed reaction selected from esterification of a carboxylic acid with an alcohol, liquefaction of corn or other cereals, saccharification of corn or other cereals to convert starch into sugars, isomerization of glucose to fructose, synthesis of chiral compounds, interesterification of oils, degumming oil, treating wastewater, clarifying fruit juice, producing glucose by the starch process, producing glucose and galactose from lactose, synthesizing compounds having peptide bonds, producing 6-aminopenicillic acid from penicillin G, converting sugars to alcohol, removing sulfur from petroleum fractions, converting acrylonitrile to acrylamide, converting 3-cyanopyridine to nicotinamide or degrading stains in a laundry soil. The improvement comprises immobilizing the enzyme in an enzyme immobilization material that immobilizes and stabilizes the enzyme and is permeable to a compound smaller than the enzyme.

Other aspects are uses of an immobilized enzyme for detecting an analyte wherein the enzyme is immobilized in an enzyme immobilization material. The enzyme immobilization material immobilizes and stabilizes the enzyme and is permeable to a compound smaller than the enzyme. The analyte detected comprises urea, uric acid, cholesterol, drugs, peroxide, dissolved gases (e.g., oxygen, carbon dioxide, etc.) a pesticide, a toxin, or a microbe.

Yet other aspects are uses of an immobilized enzyme for separation or removal of a substance from a mixture wherein the enzyme is immobilized in an enzyme immobilization material. The enzyme immobilization material immobilizes and stabilizes the enzyme and is permeable to a compound smaller than the enzyme.

Further aspects are uses of an immobilized enzyme in a chemically active film surface that reacts with at least one substance contacting the film surface wherein the enzyme is immobilized in an enzyme immobilization material. The enzyme immobilization material immobilizes and stabilizes the enzyme and is permeable to a compound smaller than the enzyme.

Other objects and features will be in part apparent and in part pointed out hereinafter.

### BRIEF DESCRIPTION OF THE DRAWINGS

FIG. 1 is a fluorescence micrograph of a low molecular weight alginate modified with tetrapentylammonium ions.

FIG. 2 is a series of fluorescence micrographs of the formation of NADH at alcohol dehydrogenase immobilized in a modified Nafion® membranes that were treated with NAD⁺ and ethanol in a pH 7.15 phosphate buffer where (a) is an unmodified Nafion® membrane, (b) is a tetramethylammonium bromide/Nafion® membrane, (c) is a tetraethylammonium bromide/Nafion® membrane, (d) is a tetrapropylammonium bromide/Nafion® membrane, (e) is a tetrabutylammonium bromide/Nafion® membrane, and (f) is a tetrapentylammonium bromide/Nafion® membrane.

FIG. 3 is a fluorescence micrograph of annealed, alcohol dehydrogenase immobilized in a tetrabutylammonium bromide/Nafion® membrane that was treated with a NAD⁺ and ethanol solution in pH 7.15 phosphate buffer.

FIG. 4 is a fluorescence micrograph of aldehyde dehydrogenase immobilized in a tetrapentylammonium bromide/Nafion® membrane that was treated with a NAD⁺ and acetaldehyde solution in pH 7.15 phosphate buffer.

FIG. 5 is a graph showing the current vs. time curve for an ethanol sensor.

FIG. 6 shows representative fluorescence micrographs of hydrophobically modified chitosan in Ru(bpy)₃⁺².

FIG. 7 shows representative fluorescence micrographs of hydrophobically modified chitosan membranes soaked in fluorescein isothiocyanate (FITC).

FIG. 8 shows the KD^{1/2} values for flux of caffeine through hydrophobically modified chitosan as a function of the alkyl chain length of the modifier and the solvent in which the polymer is resuspended.

FIG. 9 shows KD^{1/2} values for transport of Ru(bpy)₃⁺² through hydrophobically modified chitosan membranes.

FIG. 10 is a schematic representation of a particle coated with an electron mediator, an enzyme (with two subunits), and an enzyme immobilization material.

FIG. 11 is a schematic of an airbrush spray drying a mixture onto a polycarbonate shield.

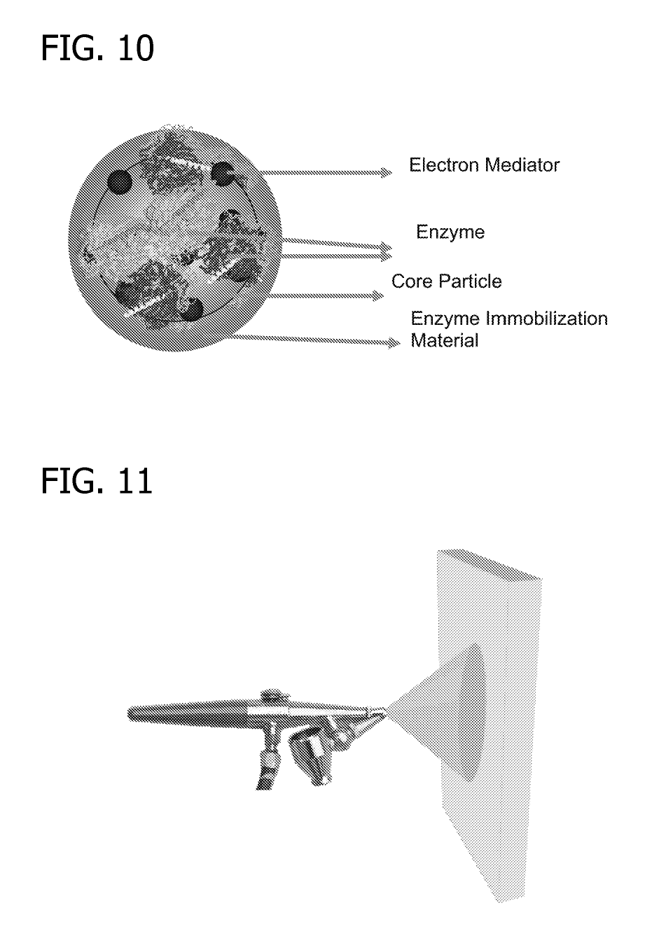

FIG. 12 is a linear sweep voltammogram demonstrating the retention of activity of encapsulated alcohol dehydrogenase as described in Example 11 and made into a carbon composite electrode.

FIG. 13 is a graph of the relative enzyme activity for starch-consuming amylase immobilized in various enzyme immobilization materials.

FIG. 14 is a graph of the relative enzyme activity for maltose-consuming amylase immobilized in various enzyme immobilization materials.

FIG. 15 is a graph of the enzyme activity after 24 hours of incubation at room temperature for free lipase, Novozym® 435 immobilized lipase, and hexyl-modified chitosan immobilized lipase.

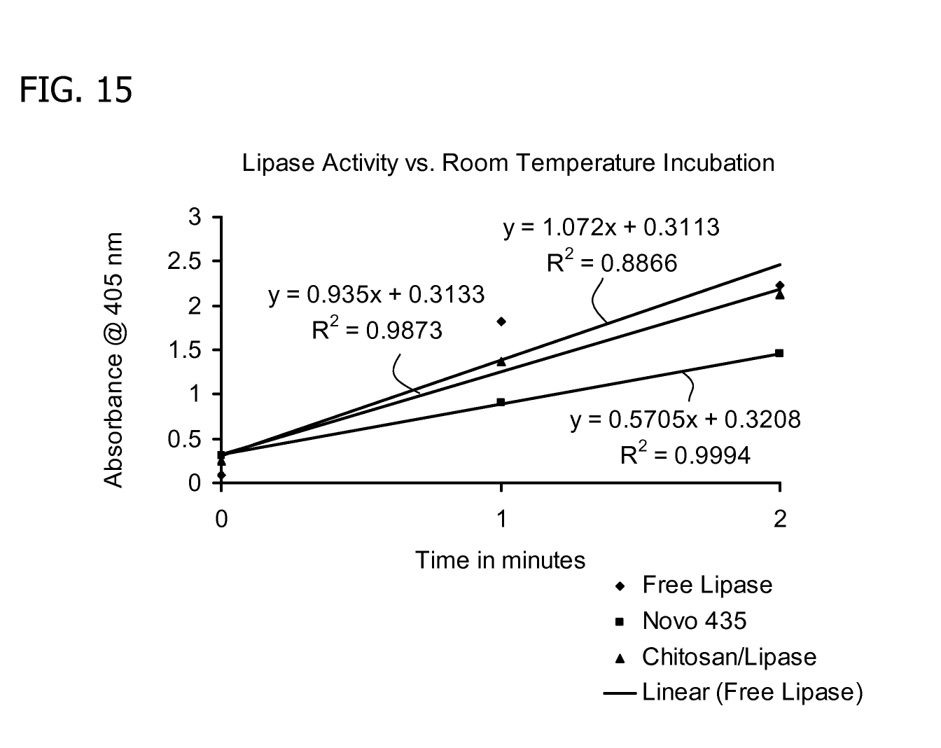

FIG. 16 is a graph of the enzyme activity after 24 hours of incubation at 65° C. for free lipase, Novozym® 435 immobilized lipase, and hexyl-modified chitosan immobilized lipase.

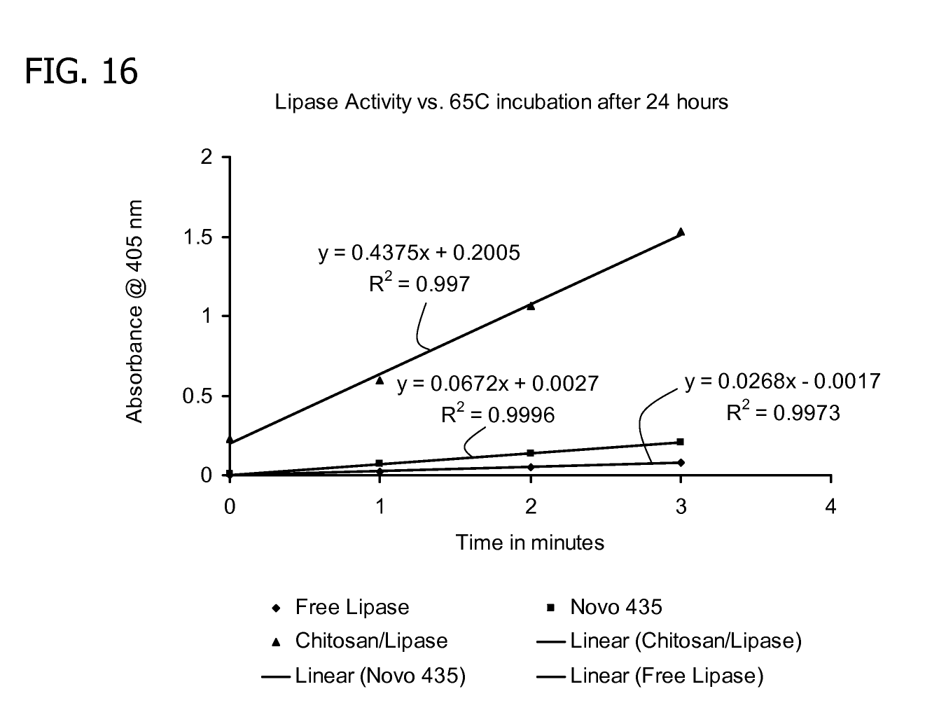

FIG. 17 is a graph of the enzyme activity after 20 hours of incubation in 50 mM NaOH solution for free lipase, Novozym® 435 immobilized lipase, and tetraethylammonium-modified Nafion® immobilized lipase.

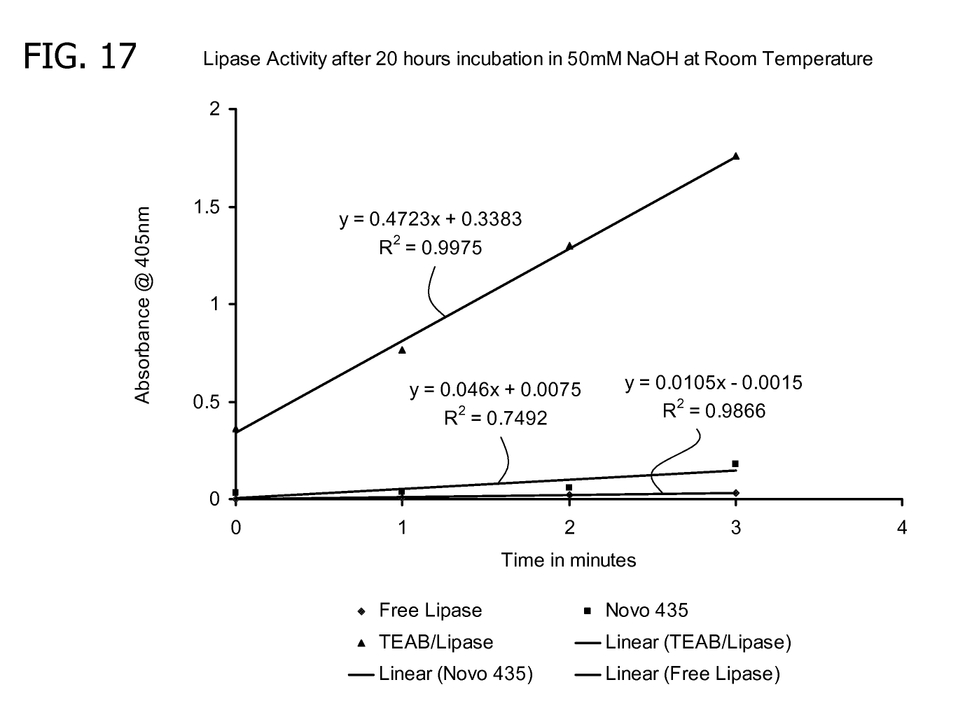

FIG. 18 is a graph of the enzyme activity after 24 hours of incubation in 50 mM NaOH solution for free lipase, Novozym® 435 immobilized lipase, and hexyl-modified chitosan immobilized lipase.

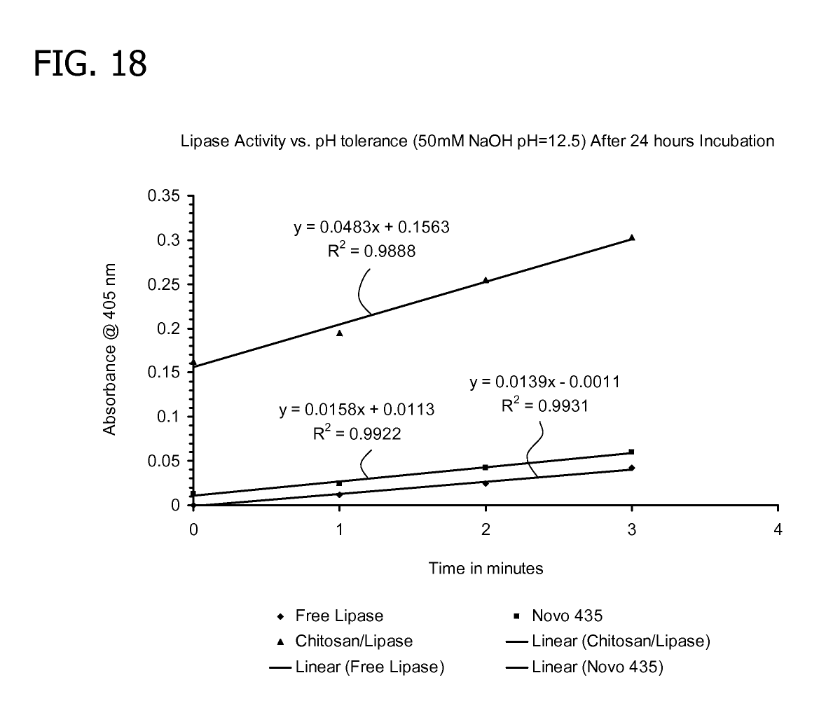

FIG. 19 is a graph of the potential versus the current density using a half cell having the laccase electrode at the cathode.

### DETAILED DESCRIPTION OF THE INVENTION

Applicants have identified new uses for enzymes immobilized in an enzyme immobilization material that immobilizes and stabilizes the enzyme. The enzyme immobilization materials described herein significantly stabilize the immobilized enzymes to denaturation or other degradation. In some embodiments, the enzymes are stabilized such that the enzymes retain at least about 75% of their catalytic activity for at least 7 days to 1095 days or more. In various embodiments, the enzymes are stabilized such that the enzymes retain at least about 75% of their catalytic activity for at least 30 days to 1095 days or more. In embodiments in which the enzyme is subjected to high temperatures (i.e., a temperature of at least about 65° C.), the enzymes are stabilized such that when exposed to a temperature of at least about 65° C. for at least about 18 hours, they retain at least about 12% of an activity of an otherwise identical free enzyme exposed to room temperature for the same amount of time. Further, in other embodiments, the enzymes are stabilized such that when exposed to a temperature of at least about 95° C. for at least about 18 hours, they retain at least about 1% of an activity of an otherwise identical free enzyme exposed to room temperature for the same amount of time. In instances where the enzyme is subjected to highly alkaline conditions (i.e., pH greater than 12.5), the enzymes are stabilized such that when exposed to a pH of at least about 12.5, they retain at least about 9% of an activity of an otherwise identical free enzyme exposed to its optimal pH for the same amount of time. These enzyme immobilization materials have pore and channel sizes that retain the enzyme within the pores while allowing various substances smaller than the enzyme to contact the enzyme within the pore. These enzyme immobilization materials can have pore size diameters ranging from 6 nm to 1 μm, can operate at a pH range of 0 to 14 can allow for channel size alteration to change substrate diffusion or selectivity, and can maintain enzyme immobilization material stability and enzyme activity in multiple solvents.

Immobilizing enzymes of the invention can make the enzymes reusable, stabilize the enzyme to changes in pH or temperature, allow enzymes to be removed easily from solution to stop a chemical reaction quickly, simplify reaction product purification, or minimize product contamination with the enzyme. The benefits arising from these advantages are generally lower enzyme cost (e.g., due to repetitive use), enhanced process control, and less costly purification steps.

As described in more detail below, the various uses for immobilized enzymes generally are as catalysts for chemical transformations, as catalysts for detecting various analytes, and for catalysts used in various separation and purification processes.

As described in more detail herein, the immobilized enzymes can be in the form of a film, a particle, a coating on a substrate (e.g., mesh, wire, electrode, inert particle, etc.) and the like.

For purposes of this invention, “free enzyme” is an enzyme that is dissolved in a buffer solution. A free enzyme is not immobilized. Also, the optimal pH of an enzyme is the pH where it is known to have optimal activity. Where that optimal pH is a range, the optimal pH is the midpoint of that range ±0.2 pH units.

### Immobilized Enzyme or Organelle Particles

Various aspects of the present invention are directed to a particle comprising a core coated with an immobilized enzyme. The core can be a material that provides a support for the immobilized enzyme layer that is coated on the core. The immobilized enzyme layer comprises an enzyme, an enzyme immobilization material, and an optional electron mediator. The components of these particles are described in more detail below.

Another of the various aspects of the present invention is a process for preparing a particle coated with an immobilized enzyme. This process comprises mixing a solution comprising an enzyme with a suspension comprising at least one support particle, an immobilization material, and a liquid medium to form a mixture. This mixture is then spray-dried to produce the coated particles.

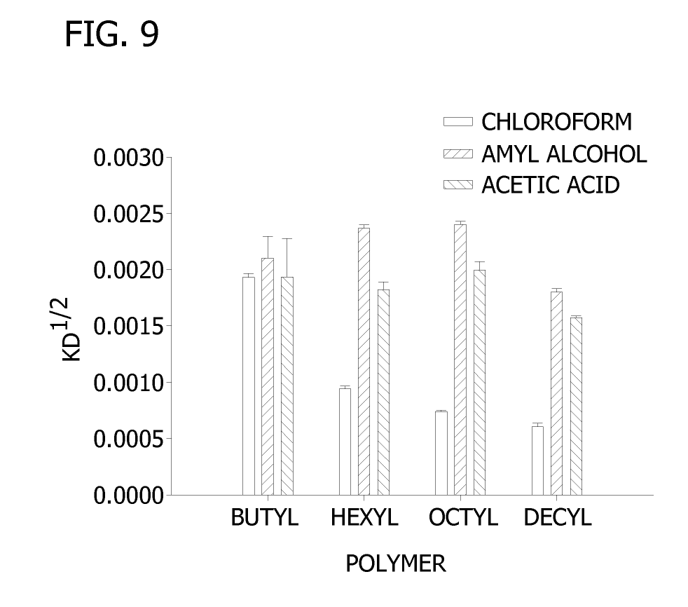

The particle produced can include a core, an optional electron mediator, an enzyme, and an enzyme immobilization material (e.g., polymer matrix) as shown in FIG. 10. The polymer matrix, which functions to stabilize the enzyme and fix it to the support, can be the various enzyme immobilization materials described below. Additionally, various compounds can be added in addition to the enzyme in the matrix that aid enzyme function. For example, electron mediators, cofactors, and coenzymes can be immobilized and will not leach into a liquid upon contact or repeated washes.

In various preferred embodiments, the enzyme is not covalently attached or adsorbed to the core. Further, preferably, the enzyme does not leach from the enzyme immobilization material into a liquid medium that the immobilized enzyme layer contacts. Typically, the immobilized enzyme particles comprise from about 0.1 wt. % to about 25 wt. % of the core and about 0.1 wt. % to about 70 wt. % of the coating and the coating comprises from about 0.1 wt. % to about 29 wt. % of the enzyme, about 0.1 wt. % to about 43 wt. % of the enzyme immobilization material, up to about 29 wt. % of the electron mediator. Typically, the total weight percent of the enzyme and the electron mediator can be up to 57 wt. % of the coating.

### Enzymes

An enzyme is used to catalyze a desired reaction. Generally, naturally-occurring enzymes, man-made enzymes, artificial enzymes and modified naturally-occurring enzymes can be immobilized. In addition, engineered enzymes that have been engineered by natural or directed evolution can be used. Stated another way, an organic or inorganic molecule that mimics an enzyme's properties can be used in embodiments of the present invention. Although these engineered enzymes may have greater stability as compared to the same enzyme that has not undergone natural or directed evolution, the engineered enzyme is further stabilized in the manner described herein by being immobilized by the enzyme immobilization material of the invention. Therefore, an engineered enzyme that is immobilized in or on a material which does not also stabilize the enzyme is not within the scope of the present invention. The enzymes that can be immobilized are oxidoreductases, transferases, hydrolases, lyases, isomerases, ligases, or combinations thereof. Other enzymes that can be used can be obtained by commonly used recombinant genetic methods such as error-prone PCR and gene shuffling. Furthermore, other suitable enzymes may be obtained by the mining of DNA from various environments such as in soil.

In various preferred embodiments, enzymes immobilized are lipases, glucose isomerases, nitrilases, glucose oxidases, proteases (e.g., pepsin), amylases (e.g., fungal amylase, maltogenic amylase), cellulases, lactases, esterases, carbohydrases, hemicellulases, pentosanases, xylanases, pullulanases, β-glucanases, acetolactate decarboxylases, β-glucosidases, glutaminases, penicillin acylases, chloroperoxidases, aspartic β-decarboxylases, cyclodextrin glycosyltransferases, subtilisins, aminoacylases, alcohol dehydrogenases, amino acid oxidases, phospholipases, ureases, cholesterases, desulfinases, lignin peroxidases, pectinases, oxidoreductases, dextranases, glucosidases, galactosidases, glucoamylases, maltases, sucrases, invertases, naringanases, bromelain, ficin, papain, pepsins, peptidases, chymosin, thermolysins, trypsins, triglyceridases, pregastric esterases, phosphatases, phytases, amidases, glutaminases, lysozyme, catalases, dehydrogenases, peroxidases, lyases, fumarases, histadases, aminotransferases, ligases, cyclases, racemases, mutases, oxidases, reductases, ligninases, laccases, chloroperoxidases, haloperoxidases, hydrogenases, nitrogenases, oxynitrilases (mandelonitrile lyases), or combinations thereof.

In various embodiments, the enzyme catalyzes reactions wherein glucose is produced. In one system, β-glucosidase can be used to hydrolyze cellobiose to glucose. Further, cellulases catalyze the hydrolysis of cellulose to glucose and amylases catalyze the hydrolysis of starch or maltose to glucose. Complex carbohydrates are the most abundant biological molecules and are a good source of substrate, but glucose has a wider range of uses than complex carbohydrates, so the carbohydrates are preferably broken down to low-molecular weight components, like glucose. Cellulose is the most abundant complex carbohydrate and it is formed from glucose sub-units. It is easily broken down by cellulases that hydrolyze the glycosidic bonds. Bioreforming of complex substrates to their low-molecular weight components can be achieved by catalysis with enzymes. These enzymes can be used for the digestion of polysaccharides (starch and cellulose) and disaccharides (sucrose and lactose) to individual carbohydrates that can be used in a larger number of reactions.

### Organelles

An enzyme or group of enzymes in an organelle can catalyze various reactions. These include the oxidation of substrates. Any organelle that contains enzymes and/or enzymes and electron mediators that catalyze desired chemical transformations can be immobilized and coated on a core particle to prepare the particles of the invention. Specifically, glyoxysome, peroxisome, mitochondria, mitoplasts, thylakoids, chloroplasts, hydrogenosomes, and combinations thereof can be immobilized and used to prepare the particles coated with immobilized organelles. In various preferred embodiments, the organelle is mitochondria, mitoplasts, thylakoids, chloroplasts, or combinations thereof.

The organelles contain various enzymes. For example, mitochondria or mitoplasts contain the enzymes necessary for the citric acid cycle that oxidize pyruvate produced by glycolysis to ATP, NADH, FADH₂ and CO₂. Mitoplasts are mitochondria with the outer membrane removed. Generally, these mitochondrial enzymes are aconitase, fumarase, malate dehydrogenase, succinate dehydrogenase, succinyl-CoA synthetase, isocitrate dehydrogenase, ketoglutarate dehydrogenase, and citrate synthase. Mitochondria and mitoplasts contain the enzymes and coenzymes of the Kreb's cycle and the electron transport chain, so they are ideally designed for completely oxidizing common fuel fluids, but unlike a microbe, they have fewer transport limitations due to smaller diffusion lengths, no biofilm formation, and no need to transport fuel across the cell wall.

The enzymes contained in glyoxysome include malate synthase, malate dehydrogenase, citrate (Si)-synthase, aconitate hydratase, and isocitrate lyase. Further the enzymes contained in peroxisomes include catalase, D-amino acid oxidase, and uric acid oxidase.

Generally, the enzymes contained within the chloroplast include RuBisCO, phosphoglycerate kinase, G3P dehydrogenase, triose phosphate isomerase, aldolase, fructose-1,6-bisphosphatase, transketolase, S1,7BPase, epimerase, ribose isomerase, and phosphoribulokinase. The enzymes within the hydrogenosome include succinyl-CoA synthetase.

Organelles, particularly mitochondria, can be more difficult to immobilize than isolated enzymes. The organelles require a particular range of osmotic pressures that mimic those in the cell, so the organelle will not either expand or contract to an extent that damages the organelle. Also, because the organelle used typically has a membrane, the mass transport of the fuel fluid to the enzyme(s) that catalyze the oxidation is maximized, so the current density of the biofuel cell does not decrease.

In some of the various embodiments, the organelles are isolated from the cell. Stated another way, these organelles are extracted from the cell and contain the contents of the specific organelle surrounded by its particular membrane only. For example, when the immobilized organelle is mitochondria, the distinct mitochondria organelle with its two membranes is immobilized within the immobilization material. These organelles are not contained within or immobilized within a cell.

### Electron Mediators

The electron mediator serves to accept or donate electron(s), readily changing from oxidized to reduced forms. Electron mediators are used with organelles and oxidoreductase enzymes for chemical transformations wherein the transfer of electrons is mediated. Various enzymes can also require cofactors such as magnesium or calcium ions in order for the enzyme to be active. The electron mediator is a compound that can be incorporated into the immobilization material. Exemplary electron mediators are nicotinamide adenine dinucleotide (NAD⁺), flavin adenine dinucleotide (FAD), nicotinamide adenine dinucleotide phosphate (NADP), pyrroloquinoline quinone (PQQ), equivalents of each, and combinations thereof. Other exemplary electron mediators are phenazine methosulfate, dichlorophenol indophenol, short chain ubiquinones, potassium ferricyanide, a protein, a metalloprotein, stellacyanin, a transition metal complex of osmium, ruthenium, iron, nickel, rhodium, rhenium, or cobalt with 1,10-phenanthroline (phen), 2,2′-bipyridine (bpy) or 2,2′,2″-terpyridine (terpy), methylene green, methylene blue, poly(methylene green), poly(methylene blue), luminol, nitro-fluorenone derivatives, azines, osmium phenanthrolinedione, catechol-pendant terpyridine, toluene blue, cresyl blue, nile blue, neutral red, phenazine derivatives, tionin, azure A, azure B, toluidine blue O, acetophenone, metallophthalocyanines, nile blue A, modified transition metal ligands, 1,10-phenanthroline-5,6-dione, 1,10-phenanthroline-5,6-diol, [Re(phen-dione)(CO)₃Cl], [Re(phen-dione)₃](PF₆)₂, poly(metallophthalocyanine), poly(thionine), quinones, diimines, diaminobenzenes, diaminopyridines, phenothiazine, phenoxazine, toluidine blue, brilliant cresyl blue, 3,4-dihydroxybenzaldehyde, poly(acrylic acid), poly(azure I), poly(nile blue A), polyaniline, polypyridine, polypyrole, polythiophene, poly(thieno[3,4-b]thiophene), poly(3-hexylthiophene), poly(3,4-ethylenedioxypyrrole), poly(isothianaphthene), poly(3,4-ethylenedioxythiophene), poly(difluoroacetylene), poly(4-dicyanomethylene-4H-cyclopenta[2,1-b;3,4-b′]dithiophene), poly(3-(4-fluorophenyl)thiophene), poly(neutral red), or combinations thereof. Alternatively, a reversible redox couple that has a standard reduction potential of 0.0V±0.5 V is used as the electron mediator.

### Enzyme or Organelle Immobilization Materials

An enzyme or organelle immobilization material can be used to immobilize and stabilize enzymes or organelles. This discussion of immobilization materials applies to enzyme immobilization materials as well as organelle immobilization materials. In various embodiments, the enzyme immobilization material is permeable to a compound smaller than the enzyme or organelle so the desired reaction can be catalyzed by the immobilized enzyme or organelle.

Generally, an enzyme is used to catalyze various reactions and this enzyme is immobilized in an enzyme immobilization material that both immobilizes and stabilizes the enzyme. For purposes of the present invention, an enzyme or organelle is “stabilized” if it either: (1) retains at least about 75% of its initial catalytic activity for at least about 30 days when continuously catalyzing a chemical transformation; (2) retains at least about 75% of its initial catalytic activity for at least about 5 days when continuously catalyzing a chemical transformation; (3) when exposed to a temperature of at least about 65° C. for at least about 18 hours, it retains at least about 12% of an activity of an otherwise identical free enzyme exposed to room temperature for the same amount of time; (4) when exposed to a temperature of at least about 95° C. for at least about 18 hours, it retains at least about 1% of an activity of an otherwise identical free enzyme exposed to room temperature for the same amount of time; (5) when exposed to a pH of greater than about 12.5 for at least about 18 hours, it retains at least about 9% of an activity of an otherwise identical free enzyme exposed to its optimal pH for the same amount of time; or (6) when exposed to a pH of less than about 2, the stabilized enzyme retains at least about 10% of its initial bioelectrocatalytic activity for at least about 1 hour when continuously catalyzing a chemical transformation. Typically, a free enzyme in solution loses its catalytic activity within a few hours to a few days, whereas a properly immobilized and stabilized enzyme can retain its catalytic activity for at least about 5 days to about 1095 days. Thus, the immobilization of the enzyme provides a significant advantage in stability. The retention of catalytic activity is defined as the enzyme or organelle having at least about 75% of its initial activity, which can be measured by chemiluminescence, electrochemical, UV-Vis, radiochemical, or fluorescence assay wherein the intensity of the property is measured at an initial time. Typically, a fluorescence assay is used to measure the enzyme or organelle activity. In various embodiments, the enzyme or organelle retains at least about 75% of its initial activity while the enzyme or organelle is continuously catalyzing a chemical transformation.

With respect to the stabilization of the enzyme or organelle, the enzyme immobilization material provides a chemical and mechanical barrier to prevent or impede enzyme denaturation. To this end, the enzyme immobilization material physically confines the enzyme, preventing the enzyme from unfolding. The process of unfolding an enzyme from a folded three-dimensional structure is one mechanism of enzyme denaturation.

In some embodiments, the enzyme immobilization material stabilizes the enzyme or organelle so that the enzyme or organelle retains its catalytic activity for at least about 5 days to about 730 days. In other embodiments, the immobilized enzyme or organelle retains at least about 75% of its initial catalytic activity for at least about 30, 45, 60, 75, 90, 105, 120, 150, 180, 210, 240, 270, 300, 330, 365, 400, 450, 500, 550, 600, 650, 700, 730, 800, 850, 900, 950, 1000, 1050, 1095 days or more. In various embodiments, the immobilized enzyme or organelle retains at least about 75%, 80%, 85%, 90%, 95% or more of its initial catalytic activity for at least about 5, 7, 10, 15, 20, 25, 30, 45, 60, 75, 90, 105, 120, 150, 180, 210, 240, 270, 300, 330, 365, 400, 450, 500, 550, 600, 650, 700, 730, 800, 850, 900, 950, 1000, 1050, 1095 days or more.

In various embodiments, an enzyme having greater temperature or pH stability may also retain at least about 75% of its initial catalytic activity for at least about 5 days when actively catalyzing a chemical transformation as described above.

In other embodiments, when exposed to a pH of less than about 2, the stabilized enzyme retains at least about 10, 15, 20, 25, 30, 35, 40, 45, 50, 55, 60, 65, 70, 75, 80, 85, 90 or 95% of its initial bioelectrocatalytic activity for at least about 1, 2, 3, 4, 8, 12, 16, 20, 24, 36, 48, 60, 72, 96, 120, 144, 168, 192, 216 or 240 hours, or more when continuously catalyzing a chemical transformation. More specifically, the stabilized enzyme may retain a percentage of its initial bioelectrocatalytic activity for at least the period of time as specified in the following table when continuously catalyzing a chemical transformation, wherein an “X” in the table below signifies a selection of a specific percentage of initial bioelectrocatalytic activity retained by the stabilized enzyme over at least the specified time period.

| Time | % of Initial Bioelectrocatalytic Activity | | | | | | | | | | | | | | | | | |
|---|---|---|---|---|---|---|---|---|---|---|---|---|---|---|---|---|---|---|
| (Hours) | 10 | 15 | 20 | 25 | 30 | 35 | 40 | 45 | 50 | 55 | 60 | 65 | 70 | 75 | 80 | 85 | 90 | 95 |
| 1 | X | X | X | X | X | X | X | X | X | X | X | X | X | X | X | X | X | X |
| 2 | X | X | X | X | X | X | X | X | X | X | X | X | X | X | X | X | X | X |
| 3 | X | X | X | X | X | X | X | X | X | X | X | X | X | X | X | X | X | X |
| 4 | X | X | X | X | X | X | X | X | X | X | X | X | X | X | X | X | X | X |
| 8 | X | X | X | X | X | X | X | X | X | X | X | X | X | X | X | X | X | X |
| 12 | X | X | X | X | X | X | X | X | X | X | X | X | X | X | X | X | X | X |
| 16 | X | X | X | X | X | X | X | X | X | X | X | X | X | X | X | X | X | X |
| 20 | X | X | X | X | X | X | X | X | X | X | X | X | X | X | X | X | X | X |
| 24 | X | X | X | X | X | X | X | X | X | X | X | X | X | X | X | X | X | X |
| 36 | X | X | X | X | X | X | X | X | X | X | X | X | X | X | X | X | X | X |
| 48 | X | X | X | X | X | X | X | X | X | X | X | X | X | X | X | X | X | X |
| 72 | X | X | X | X | X | X | X | X | X | X | X | X | X | X | X | X | X | X |
| 96 | X | X | X | X | X | X | X | X | X | X | X | X | X | X | X | X | X | X |
| 120 | X | X | X | X | X | X | X | X | X | X | X | X | X | X | X | X | X | X |
| 144 | X | X | X | X | X | X | X | X | X | X | X | X | X | X | X | X | X | X |
| 168 | X | X | X | X | X | X | X | X | X | X | X | X | X | X | X | X | X | X |
| 192 | X | X | X | X | X | X | X | X | X | X | X | X | X | X | X | X | X | X |
| 216 | X | X | X | X | X | X | X | X | X | X | X | X | X | X | X | X | X | X |
| 240 | X | X | X | X | X | X | X | X | X | X | X | X | X | X | X | X | X | X |

Also, in various embodiments, after immobilization in the enzyme immobilization material, the enzyme retains at least about 0.65, 0.7, 0.75, 0.8, 0.85, 0.9, 0.95, or more, of its initial activity relative to the activity of the enzyme before immobilization.

An immobilized enzyme is an enzyme that is physically confined in a certain region of the enzyme immobilization material while retaining its catalytic activity. There are a variety of methods for enzyme immobilization, including carrier-binding, cross-linking and entrapping. Carrier-binding is the binding of enzymes to water-insoluble carriers. Cross-linking is the intermolecular cross-linking of enzymes by bifunctional or multifunctional reagents. Entrapping is incorporating enzymes into the lattices of a semipermeable material. The particular method of enzyme immobilization is not critically important, so long as the enzyme immobilization material (1) immobilizes the enzyme, and (2) stabilizes the enzyme. In various embodiments, the enzyme immobilization material is also permeable to a compound smaller than the enzyme.

An immobilized organelle is an organelle that is physically confined in a certain region of the immobilization material while retaining its activity.

With reference to the immobilization material's permeability to various compounds that are smaller than an enzyme or organelle, the immobilization material allows the movement of a substrate, fuel fluid, or oxidant compound through it so the compound can contact the enzyme or organelle. The immobilization material can be prepared in a manner such that it contains internal pores, channels, openings or a combination thereof, which allow the movement of the compound throughout the immobilization material, but which constrain the enzyme or organelle to substantially the same space within the immobilization material. Such constraint allows the enzyme or organelle to retain its catalytic activity. In various preferred embodiments, the enzyme or organelle is confined to a space that is substantially the same size and shape as the enzyme or organelle, wherein the enzyme or organelle retains substantially all of its catalytic activity. The pores, channels, or openings have physical dimensions that satisfy the above requirements and depend on the size and shape of the specific enzyme or organelle to be immobilized.

In one embodiment, the enzyme or organelle is preferably located within a pore of the immobilization material and the compound travels in and out of the immobilization material through transport channels. The relative size of the pores and transport channels can be such that a pore is large enough to immobilize an enzyme or organelle, but the transport channels are too small for the enzyme or organelle to travel through them. Further, a transport channel preferably has a diameter of at least about 10 nm. In still another embodiment, the pore diameter to transport channel diameter ratio is at least about 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1 or more. In yet another embodiment, preferably, a transport channel has a diameter of at least about 2 nm and the pore diameter to transport channel diameter ratio is at least about 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1 or more.

In some of the embodiments, the immobilization material has a micellar or inverted micellar structure. Generally, the molecules making up a micelle are amphipathic, meaning they contain a polar, hydrophilic group and a nonpolar, hydrophobic group. The molecules can aggregate to form a micelle, where the polar groups are on the surface of the aggregate and the hydrocarbon, nonpolar groups are sequestered inside the aggregate. Inverted micelles have the opposite orientation of polar groups and nonpolar groups. The amphipathic molecules making up the aggregate can be arranged in a variety of ways so long as the polar groups are in proximity to each other and the nonpolar groups are in proximity to each other. Also, the molecules can form a bilayer with the nonpolar groups pointing toward each other and the polar groups pointing away from each other. Alternatively, a bilayer can form wherein the polar groups can point toward each other in the bilayer, while the nonpolar groups point away from each other.

Modified Nafion®

In one preferred embodiment, the micellar immobilization material is a modified perfluoro sulfonic acid-PTFE copolymer (or modified perfluorinated ion exchange polymer) (modified Nafion® or modified Flemion®) membrane. The perfluorinated ion exchange polymer membrane is modified with a hydrophobic cation that is larger than the ammonium (NH₄⁺) ion. The hydrophobic cation serves the dual function of (1) dictating the membrane's pore size and (2) acting as a chemical buffer to help maintain the pore's pH level, both of which stabilize the enzyme.

With regard to the first function of the hydrophobic cation, mixture-casting a perfluoro sulfonic acid-PTFE copolymer (or perfluorinated ion exchange polymer) with a hydrophobic cation to produce a modified perfluoro sulfonic acid-PTFE copolymer (or modified perfluorinated ion exchange polymer) (Nafion® or Flemion®) membrane provides an immobilization material wherein the pore size is dependent on the size of the hydrophobic cation. Accordingly, the larger the hydrophobic cation, the larger the pore size. This function of the hydrophobic cation allows the pore size to be made larger or smaller to fit a specific enzyme or organelle by varying the size of the hydrophobic cation.

Regarding the second function of the hydrophobic cation, the properties of the perfluoro sulfonic acid-PTFE copolymer (or perfluorinated ion exchange polymer) membrane are altered by exchanging the hydrophobic cation for protons as the counterion to the —SO₃⁻ groups on the perfluoro sulfonic acid-PTFE copolymer (or anions on the perfluorinated ion exchange polymer) membrane. This change in counterion provides a buffering effect on the pH because the hydrophobic cation has a much greater affinity for the —SO₃⁻ sites than protons do. This buffering effect of the membrane causes the pH of the pore to remain substantially unchanged with changing solution pH; stated another way, the pH of the pore resists changes in the solution's pH. In addition, the membrane provides a mechanical barrier, which further protects the immobilized enzymes or organelles.

In order to prepare a modified perfluoro sulfonic acid-PTFE copolymer (or perfluorinated ion exchange polymer) membrane, the first step is to cast a suspension of perfluoro sulfonic acid-PTFE copolymer (or perfluorinated ion exchange polymer), particularly Nafion®, with a solution of the hydrophobic cations to form a membrane. The excess hydrophobic cations and their salts are then extracted from the membrane, and the membrane is re-cast. Upon re-casting, the membrane contains the hydrophobic cations in association with the —SO₃⁻ sites of the perfluoro sulfonic acid-PTFE copolymer (or perfluorinated ion exchange polymer) membrane. Removal of the salts of the hydrophobic cation from the membrane results in a more stable and reproducible membrane; if they are not removed, the excess salts can become trapped in the pore or cause voids in the membrane.

In one embodiment, a modified Nafion® membrane is prepared by casting a suspension of Nafion® polymer with a solution of a salt of a hydrophobic cation such as quaternary ammonium bromide. Excess quaternary ammonium bromide or hydrogen bromide are removed from the membrane before it is re-cast to form the salt-extracted membrane. Salt extraction of membranes retains the presence of the quaternary ammonium cations at the sulfonic acid exchange sites, but eliminates complications from excess salt that may be trapped in the pore or may cause voids in the equilibrated membrane. The chemical and physical properties of the salt-extracted membranes have been characterized by voltammetry, ion exchange capacity measurements, and fluorescence microscopy before enzyme immobilization. Exemplary hydrophobic cations are ammonium-based cations, quaternary ammonium cations, alkyltrimethylammonium cations, alkyltriethylammonium cations, organic cations, phosphonium cations, triphenylphosphonium, pyridinium cations, imidazolium cations, hexadecylpyridinium, ethidium, viologens, methyl viologen, benzyl viologen, bis(triphenylphosphine)iminium, metal complexes, bipyridyl metal complexes, phenanthroline-based metal complexes, [Ru(bipyridine)₃]²⁺ and [Fe(phenanthroline)₃]³⁺.

In one preferred embodiment, the hydrophobic cations are ammonium-based cations. In particular, the hydrophobic cations are quaternary ammonium cations. In another embodiment, the quaternary ammonium cations are represented by Formula 1:

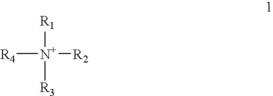

wherein R₁, R₂, R₃, and R₄ are independently hydrogen, hydrocarbyl, substituted hydrocarbyl, or heterocyclo wherein at least one of R₁, R₂, R₃, and R₄ is other than hydrogen. In a further embodiment, preferably, R₁, R₂, R₃, and R₄ are independently hydrogen, methyl, ethyl, propyl, butyl, pentyl, hexyl, heptyl, octyl, nonyl, decyl, undecyl, dodecyl, tridecyl or tetradecyl wherein at least one of R₁, R₂, R₃, and R₄ is other than hydrogen. In still another embodiment, R₁, R₂, R₃, and R₄ are the same and are methyl, ethyl, propyl, butyl, pentyl or hexyl. In yet another embodiment, preferably, R₁, R₂, R₃, and R₄ are butyl. In yet another embodiment, preferably, R₁, R₂, R₃, and R₄ are ethyl. Preferably, the quaternary ammonium cation is tetraethylammonium (T2A), tetrapropylammonium (T3A), tetrapentylammonium (T5A), tetrahexylammonium (T6A), tetraheptylammonium (T7A), trimethylicosylammonium (TMICA), trimethyloctyldecylammonium (TMODA), trimethylhexyldecylammonium (TMHDA), trimethyltetradecylammonium (TMTDA), trimethyloctylammonium (TMOA), trimethyldodecylammonium (TMDDA), trimethyldecylammonium (TMDA), trimethylhexylammonium (TMHA), tetrabutylammonium (TBA), triethylhexylammonium (TEHA), and combinations thereof.

### Hydrophobically Modified Polysaccharides

In other various embodiments, exemplary micellar or inverted micellar immobilization materials are hydrophobically modified polysaccharides, these polysaccharides are selected from chitosan, cellulose, chitin, starch, amylose, alginate, glycogen, and combinations thereof. In various embodiments, the micellar or inverted micellar immobilization materials are polycationic polymers, particularly, hydrophobically modified chitosan. Chitosan is a poly[β-(1-4)-2-amino-2-deoxy-D-glucopyranose]. Chitosan is typically prepared by deacetylation of chitin (a poly[β-(1-4)-2-acetamido-2-deoxy-D-glucopyranose]). The typical commercial chitosan has approximately 85% deacetylation. These deacetylated or free amine groups can be further functionalized with hydrocarbyl, particularly, alkyl groups. Thus, in various embodiments, the micellar hydrophobically modified chitosan corresponds to the structure of Formula 2

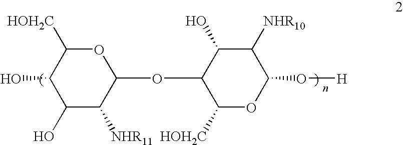

wherein n is an integer; R₁₀ is independently hydrogen, hydrocarbyl, substituted hydrocarbyl, or a hydrophobic redox mediator; and R₁₁ is independently hydrogen, hydrocarbyl, substituted hydrocarbyl, or a hydrophobic redox mediator. In certain embodiments of the invention, n is an integer that gives the polymer a molecular weight of from about 21,000 to about 500,000; preferably, from about 90,000 to about 500,000; more preferably, from about 150,000 to about 350,000; more preferably, from about 225,000 to about 275,000. In many embodiments, R₁₀ is independently hydrogen or alkyl and R₁₁ is independently hydrogen or alkyl. Further, R₁₀ is independently hydrogen or hexyl and R₁₁ is independently hydrogen or hexyl. Alternatively, R₁₀ is independently hydrogen or octyl and R₁₁ is independently hydrogen or octyl.

In other various embodiments, the micellar hydrophobically modified chitosan is a micellar hydrophobic redox mediator modified chitosan corresponding to Formula 2A

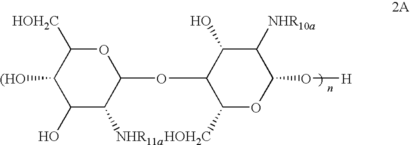

wherein n is an integer; R₁₀ₐ is independently hydrogen, or a hydrophobic redox mediator; and R₁₁ₐ is independently hydrogen, or a hydrophobic redox mediator.

Further, in various embodiments, the micellar hydrophobically modified chitosan is a modified chitosan or redox mediator modified chitosan corresponding to Formula 2B

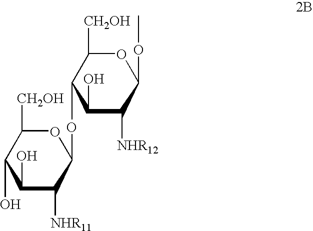

wherein R₁₁, R₁₂, and n are defined as in connection with Formula 2. In some embodiments, R₁₁ and R₁₂ are independently hydrogen or straight or branched alkyl; preferably, hydrogen, butyl, pentyl, hexyl, heptyl, octyl, nonyl, decyl, undecyl, or dodecyl. In various embodiments, R₁₁ and R₁₂ are independently hydrogen, butyl, or hexyl.

The micellar hydrophobically modified chitosans can be modified with hydrophobic groups to varying degrees. The degree of hydrophobic modification is determined by the percentage of free amine groups that are modified with hydrophobic groups as compared to the number of free amine groups in the unmodified chitosan. The degree of hydrophobic modification can be estimated from an acid-base titration and/or nuclear magnetic resonance (NMR), particularly ¹H NMR, data. This degree of hydrophobic modification can vary widely and is at least about 0.25, 0.5, 1, 2, 4, 6, 8, 10, 12, 14, 16, 18, 20, 25, 30, 32, 24, 26, 28, 40, 42, 44, 46, 48%, or more. Preferably, the degree of hydrophobic modification is from about 10% to about 45%; from about 10% to about 35%; from about 20% to about 35%; or from about 30% to about 35%.

In other various embodiments, the hydrophobic redox mediator of Formula 2A is a transition metal complex of osmium, ruthenium, iron, nickel, rhodium, rhenium, or cobalt with 1,10-phenanthroline (phen), 2,2′-bipyridine (bpy) or 2,2′,2″-terpyridine (terpy), methylene green, methylene blue, poly(methylene green), poly(methylene blue), luminol, nitro-fluorenone derivatives, azines, osmium phenanthrolinedione, catechol-pendant terpyridine, toluene blue, cresyl blue, nile blue, neutral red, phenazine derivatives, thionin, azure A, azure B, toluidine blue O, acetophenone, metallophthalocyanines, nile blue A, modified transition metal ligands, 1,10-phenanthroline-5,6-dione, 1,10-phenanthroline-5,6-diol, [Re(phen-dione)(CO)₃Cl], [Re(phen-dione)₃](PF₆)₂, poly(metallophthalocyanine), poly(thionine), quinones, diimines, diaminobenzenes, diaminopyridines, phenothiazine, phenoxazine, toluidine blue, brilliant cresyl blue, 3,4-dihydroxybenzaldehyde, poly(acrylic acid), poly(azure I), poly(nile blue A), polyaniline, polypyridine, polypyrole, polythiophene, poly(thieno[3,4-b]thiophene), poly(3-hexylthiophene), poly(3,4-ethylenedioxypyrrole), poly(isothianaphthene), poly(3,4-ethylenedioxythiophene), poly(difluoroacetylene), poly(4-dicyanomethylene-4H-cyclopenta[2,1-b;3,4-b′]dithiophene), poly(3-(4-fluorophenyl)thiophene), poly(neutral red), or combinations thereof.

Preferably, the hydrophobic redox mediator is Ru(phen)₃⁺², Fe(phen)₃⁺², Os(phen)₃⁺², Co(phen)₃⁺², Cr(phen)₃⁺², Ru(bpy)₃⁺², Os(bpy)₃⁺², Fe(bpy)₃⁺², Co(bpy)₃⁺², Cr(bpy)₃⁺², Os(terpy)₃⁺², Ru(bpy)₂(4-methyl-4′-(6-hexyl)-2,2′-bipyridine)⁺², Co(bpy)₂(4-methyl-4′-(6-hexyl)-2,2′-bipyridine)⁺², Cr(bpy)₂(4-methyl-4′-(6-hexyl)-2,2′-bipyridine)⁺², Fe(bpy)₂(4-methyl-4′-(6-hexyl)-2,2′-bipyridine)⁺², Os(bpy)₂(4-methyl-4′-(6-hexyl)-2,2′-bipyridine)⁺², or combinations thereof. More preferably, the hydrophobic redox mediator is Ru(bpy)₂(4-methyl-4′-(6-hexyl)-2,2′-bipyridine)⁺², Co(bpy)₂(4-methyl-4′-(6-hexyl)-2,2′-bipyridine)⁺², Cr(bpy)₂(4-methyl-4′-(6-hexyl)-2,2′-bipyridine)⁺², Fe(bpy)₂(4-methyl-4′-(6-hexyl)-2,2′-bipyridine)⁺², Os(bpy)₂(4-methyl-4′-(6-hexyl)-2,2′-bipyridine)⁺², or combinations thereof. In various preferred embodiments, the hydrophobic redox mediator is Ru(bpy)₂(4-methyl-4′-(6-hexyl)-2,2′-bipyridine)⁺².

For the immobilization material having a hydrophobic redox mediator as the modifier, the hydrophobic redox mediator is typically covalently bonded to the chitosan or polysaccharide backbone. Typically, in the case of chitosan, the hydrophobic redox mediator is covalently bonded to one of the amine functionalities of the chitosan through a —N—C— bond. In the case of metal complex redox mediators, the metal complex is attached to the chitosan through an —N—C— bond from a chitosan amine group to an alkyl group attached to one or more of the ligands of the metal complex. A structure corresponding to Formula 2C is an example of a metal complex attached to a chitosan

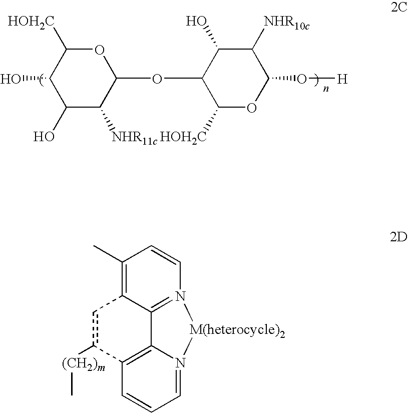

wherein n is an integer; R_{10c} is independently hydrogen or a structure corresponding to Formula 2D; R_{11c} is independently hydrogen or a structure corresponding to Formula 1D; m is an integer from 0 to 10; M is Ru, Os, Fe, Cr, or Co; and heterocycle is bipyridyl, substituted bipyridyl, phenanthroline, acetylacetone, and combinations thereof.

The hydrophobic group used to modify chitosan serves the dual function of (1) dictating the immobilization material's pore size and (2) modifying the chitosan's electronic environment to maintain an acceptable pore environment, both of which stabilize the enzyme or organelle. With regard to the first function of the hydrophobic group, hydrophobically modifying chitosan produces an immobilization material wherein the pore size is dependent on the size of the hydrophobic group. Accordingly, the size, shape, and extent of the modification of the chitosan with the hydrophobic group affects the size and shape of the pore. This function of the hydrophobic group allows the pore size to be made larger or smaller or a different shape to fit a specific enzyme or organelle by varying the size and branching of the hydrophobic group.

Regarding the second function of the hydrophobic cation, the properties of the hydrophobically modified chitosan membranes are altered by modifying chitosan with hydrophobic groups. This hydrophobic modification of chitosan affects the pore environment by increasing the number of available exchange sites to proton. In addition to affecting the pH of the material, the hydrophobic modification of chitosan provides a membrane that is a mechanical barrier, which further protects the immobilized enzymes.

Table 1 shows the number of available exchange sites to proton for the hydrophobically modified chitosan membrane.

TABLE 1
Number of available exchange sites to proton per
gram of chitosan polymer

| | Exchange sites per gram |
|---|---|
| Membrane | (×10⁻⁴ mol SO₃/g) |
| Chitosan | 10.5 ± 0.8 |
| Butyl Modified | 226 ± 21 |
| Hexyl Modified | 167 ± 45 |
| Octyl Modified | 529 ± 127 |
| Decyl Modified | 483 ± 110 |

Further, such polycationic polymers are capable of immobilizing enzymes or organelle and increasing the activity of enzymes immobilized therein as compared to the activity of the same enzyme or organelle in a buffer solution. In various embodiments, the polycationic polymers are hydrophobically modified polysaccharides, particularly, hydrophobically modified chitosan. For example, for the hydrophobic modifications noted, the enzyme activities for glucose oxidase were measured. The highest enzyme activity was observed for glucose oxidase in a hexyl modified chitosan suspended in t-amyl alcohol. These immobilization membranes showed a 2.53 fold increase in glucose oxidase enzyme activity over enzyme in buffer. Table 2 details the glucose oxidase activities for a variety of hydrophobically modified chitosans.

TABLE 2
Glucose oxidase enzyme activity for modified chitosans

| | Enzyme Activity |
|---|---|
| Membrane/Solvent | (Units/gm) |
| Buffer | 103.61 ± 3.15 |
| UNMODIFIED CHITOSAN | 214.86 ± 10.23 |
| HEXYL CHITOSAN | |
| Chloroform | 248.05 ± 12.62 |
| t-amyl alcohol | 263.05 ± 7.54 |
| 50% acetic acid | 118.98 ± 6.28 |
| DECYL CHITOSAN | |
| Chloroform | 237.05 ± 12.31 |
| t-amyl alcohol | 238.05 ± 10.02 |
| 50% acetic acid | 3.26 ± 2.82 |
| OCTYL CHITOSAN | |
| Chloroform | 232.93 ± 7.22 |
| t-amyl alcohol | 245.75 ± 9.77 |
| 50% acetic acid | 127.55 ± 11.98 |
| BUTYL CHITOSAN | |
| Chloroform | 219.15 ± 9.58 |
| t-amyl alcohol | 217.10 ± 6.55 |
| 50% acetic acid | 127.65 ± 3.02 |

To prepare the hydrophobically modified chitosans of the invention having an alkyl group as a modifier, a chitosan gel was suspended in acetic acid followed by addition of an alcohol solvent. To this chitosan gel was added an aldehyde (e.g., butanal, hexanal, octanal, or decanal), followed by addition of sodium cyanoborohydride. The resulting product was separated by vacuum filtration and washed with an alcohol solvent. The modified chitosan was then dried in a vacuum oven at 40° C. and resulted in a flaky white solid.

To prepare a hydrophobically modified chitosan of the invention having a redox mediator as a modifier, a redox mediator ligand was derivatized by contacting 4,4′-dimethyl-2,2′-bipyridine with lithium diisopropylamine followed by addition of a dihaloalkane to produce 4-methyl-4′-(6-haloalkyl)-2,2′-bipyridine. This ligand was then contacted with Ru(bipyridine)₂Cl₂ hydrate in the presence of an inorganic base and refluxed in a water-alcohol mixture until the Ru(bipyridine)₂Cl₂ was depleted. The product was then precipitated with ammonium hexafluorophosphate, or optionally a sodium or potassium perchlorate salt, followed by recrystallization. The derivatized redox mediator (Ru(bipyridine)₂(4-methyl-4′-(6-bromohexyl)-2,2′-bipyridine)⁺²) was then contacted with deacetylated chitosan and heated. The redox mediator modified chitosan was then precipitated and recrystallized.

The hydrophobically modified chitosan membranes have advantageous insolubility in ethanol. For example, the chitosan enzyme immobilization materials described above generally are functional to immobilize and stabilize the enzymes in solutions having up to greater than about 99 wt. % or 99 volume % ethanol. In various embodiments, the chitosan immobilization material is functional in solutions having 15, 20, 25, 30, 35, 40, 45, 50, 55, 60, 65, 70, 75, 80, 85, 90, 95 or more wt. % or volume % ethanol.

In other embodiments, the micellar or inverted micellar immobilization materials are polyanionic polymers, such as hydrophobically modified polysaccharides, particularly, hydrophobically modified alginate. Alginates are linear unbranched polymers containing β-(1-4)-linked D-mannuronic acid and α-(1-4)-linked L-guluronic acid residues. In the unprotonated form, β-(1-4)-linked D-mannuronic acid corresponds to the structure of Formula 3A

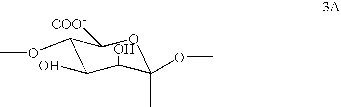

and in the unprotonated form, α-(1-4)-linked L-guluronic acid corresponds to the structure of Formula 3B

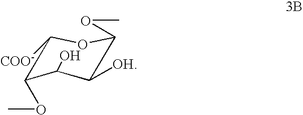

Alginate is a heterogeneous polymer consisting of polymer blocks of mannuronic acid residues and polymer blocks of guluronic acid residues.

Alginate polymers can be modified in various ways. One type is alginate modified with a hydrophobic cation that is larger than the ammonium (NH₄⁺) ion. The hydrophobic cation serves the dual function of (1) dictating the polymer's pore size and (2) acting as a chemical buffer to help maintain the pore's pH level, both of which stabilize the enzyme or organelle. With regard to the first function of the hydrophobic cation, modifying alginate with a hydrophobic cation produces an immobilization material wherein the pore size is dependent on the size of the hydrophobic cation. Accordingly, the size, shape, and extent of the modification of the alginate with the hydrophobic cation affects the size and shape of the pore. This function of the hydrophobic cation allows the pore size to be made larger or smaller or a different shape to fit a specific enzyme or organelle by varying the size and branching of the hydrophobic cation.

Regarding the second function of the hydrophobic cation, the properties of the alginate polymer are altered by exchanging the hydrophobic cation for protons as the counterion to the —CO₂⁻ groups on the alginate. This change in counterion provides a buffering effect on the pH because the hydrophobic cation has a much greater affinity for the —CO₂⁻ sites than protons do. This buffering effect of the alginate membrane causes the pH of the pore to remain substantially unchanged with changing solution pH; stated another way, the pH of the pore resists changes in the solution's pH. In addition, the alginate membrane provides a mechanical barrier, which further protects the immobilized enzymes or organelles.

In order to prepare a modified alginate membrane, the first step is to cast a suspension of alginate polymer with a solution of the hydrophobic cation to form a membrane. The excess hydrophobic cations and their salts are then extracted from the membrane, and the membrane is re-cast. Upon re-casting, the membrane contains the hydrophobic cations in association with —CO₂⁻ sites of the alginate membrane. Removal of the salts of the hydrophobic cation from the membrane results in a more stable and reproducible membrane; if they are not removed, the excess salts can become trapped in the pore or cause voids in the membrane.

In one embodiment, a modified alginate membrane is prepared by casting a suspension of alginate polymer with a solution of a salt of a hydrophobic cation such as quaternary ammonium bromide. Excess quaternary ammonium bromide or hydrogen bromide are removed from the membrane before it is re-cast to form the salt-extracted membrane. Salt extraction of membranes retains the presence of the quaternary ammonium cations at the carboxylic acid exchange sites, but eliminates complications from excess salt that may be trapped in the pore or may cause voids in the equilibrated membrane. Exemplary hydrophobic cations are ammonium-based cations, quaternary ammonium cations, alkyltrimethylammonium cations, alkyltriethylammonium cations, organic cations, phosphonium cations, triphenylphosphonium, pyridinium cations, imidazolium cations, hexadecylpyridinium, ethidium, viologens, methyl viologen, benzyl viologen, bis(triphenylphosphine)iminium, metal complexes, bipyridyl metal complexes, phenanthroline-based metal complexes, [Ru(bipyridine)₃]²⁺ and [Fe(phenanthroline)₃]³⁺.

In one preferred embodiment, the hydrophobic cations are ammonium-based cations. In particular, the hydrophobic cations are quaternary ammonium cations. In another embodiment, the quaternary ammonium cations are represented by Formula 4:

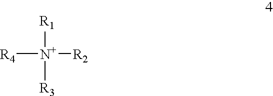

wherein R₁, R₂, R₃, and R₄ are independently hydrogen, hydrocarbyl, substituted hydrocarbyl, or heterocyclo wherein at least one of R₁, R₂, R₃, and R₄ is other than hydrogen. In a further embodiment, preferably, R₁, R₂, R₃, and R₄ are independently hydrogen, methyl, ethyl, propyl, butyl, pentyl, hexyl, heptyl, octyl, nonyl, decyl, undecyl, dodecyl, tridecyl or tetradecyl wherein at least one of R₁, R₂, R₃, and R₄ is other than hydrogen. In still another embodiment, R₁, R₂, R₃, and R₄ are the same and are methyl, ethyl, propyl, butyl, pentyl or hexyl. In yet another embodiment, preferably, R₁, R₂, R₃, and R₄ are butyl. In yet another embodiment, preferably, R₁, R₂, R₃, and R₄ are ethyl. Preferably, the quaternary ammonium cation is tetraethylammonium, tetrapropylammonium (T3A), tetrapentylammonium (T5A), tetrahexylammonium (T6A), tetraheptylammonium (T7A), trimethylicosylammonium (TMICA), trimethyloctyldecylammonium (TMODA), trimethylhexyldecylammonium (TMHDA), trimethyltetradecylammonium (TMTDA), trimethyloctylammonium (TMOA), trimethyldodecylammonium (TMDDA), trimethyldecylammonium (TMDA), trimethylhexylammonium (TMHA), tetrabutylammonium (TBA), triethylhexylammonium (TEHA), and combinations thereof.

The pore characteristics were studied and the pore structure of this membrane is ideal for enzyme immobilization, because the pores are hydrophobic, micellar in structure, buffered to external pH change, and have high pore interconnectivity.

In another experiment, ultralow molecular weight alginate and dodecylamine were placed in 25% ethanol and refluxed to produce a dodecyl-modified alginate by amidation of the carboxylic acid groups. Various alkyl amines can be substituted for the dodecylamine to produce alkyl-modified alginate having a C₄-C₁₆ alkyl group attached to varying numbers of the reactive carboxylic acid groups of the alginate structure. In various embodiments, at least about 1, 2, 4, 6, 8, 10, 12, 14, 16, 18, 20, 22, 24, 26, 28, 30, 32, 34, 36, 38, 40, 42, 44, 46, 48%, or more of the carboxylic acid groups react with the alkylamine.

The hydrophobically modified alginate membranes have advantageous insolubility in ethanol. For example, the alginate enzyme immobilization materials described above generally are functional to immobilize and stabilize the enzymes in solutions having at least about 25 wt. % or 25 volume % ethanol. In various embodiments, the alginate immobilization material is functional in solutions having 25, 30, 35, 40, 45, 50, 55, 60, 65, 70, 75, 80, 85, 90 or more wt. % or volume % ethanol.

In order to evaluate the most advantageous immobilization material for a particular enzyme or organelle, the selected enzyme or organelle can be immobilized in various immobilization materials, deposited on an electron conductor, and treated with a solution containing an electron mediator (e.g., NAD⁺) and/or a substrate for the particular enzyme in a buffer solution. A fluorescence micrograph is obtained and shows fluorescence when the enzyme or organelle immobilized in the particular immobilization material is still a catalytically active enzyme after immobilization. Enzyme activity could also be determined by any standard spectroscopic assay. This is one way to determine whether a particular immobilization material will immobilize and stabilize an enzyme or organelle while retaining the enzyme's or organelle's catalytic activity. For example, for starch consuming amylase, the enzyme immobilization material that provided the greatest relative activity is provided by immobilization of the enzyme in butyl chitosan suspended in t-amyl alcohol. For maltose consuming amylase, the greatest relative activity is provided by immobilization of the enzyme in medium molecular weight decyl modified chitosan.

### Core Component

The core is any particle that provides a support for the immobilized enzyme layer and that can be spray-dried. The core particle can be, for example, a polymer particle, a carbon particle, a zeolite particle, a metal particle, a ceramic particle, a metal oxide particle, or a combination thereof. In some embodiments, the core particle is an inert core particle. In various embodiments, the core particle is not a polymer particle. Preferred core particles do not adversely affect the stability of the enzyme or a chemical transformation involving the enzyme. In some embodiments, the core particles have an average diameter from about 200 nm to about 100 μm, depending upon the intended use of the particles when coated with the immobilized enzyme.

### Methods of Preparing Coated Particles

The coated particles are prepared by mixing a solution comprising an enzyme or organelle with a suspension comprising at least one core particle, an immobilization material, and a liquid medium and spray-drying the resulting mixture. The solution, suspension, and spray-drying step are described in more detail below.

An enzyme solution comprising the enzyme and a solvent is used in the coating procedure. Alternatively, an organelle solution comprising the organelle and a solvent is used in the coating procedure. The enzyme is combined with a solvent and mixed until a solution is formed. Acceptable enzymes and organelles are described in more detail above. The solvent can be an aqueous solution, particularly a buffer solution, such as an acetate buffer or phosphate buffer. The buffer pH is designed to provide an acceptable pH for the particular enzyme or organelle to be immobilized. Also, in various embodiments, the enzyme solution can contain an electron mediator as described above.

A suspension is prepared by combining a core particle, the desired immobilization material and a liquid medium. Exemplary core particles and immobilization materials are described above. The liquid medium can be a solvent or buffer, such as an acetate buffer or phosphate buffer. When a buffer is used as the liquid medium, the buffer pH is selected to provide an acceptable pH for the particular enzyme or organelle to be immobilized and coated.

Once the enzyme or organelle solution and the suspension are prepared, they are combined and mixed well. The resulting mixture is then dried. A preferred drying method is spray-drying because the drying also results in coating of the core particles with the immobilized enzyme layer. Conventional spray drying techniques can be used in the methods of the invention. Alternatives to spray-drying include other conventional processes for forming coated particles, such as fluidized bed granulation, spray dry granulation, rotogranulation, fluidized bed/spray drying granulation, extrusion and spheronization.

In some of the various embodiments, the solution comprises from about 0.1 wt. % to about 15 wt. % of the enzyme or organelle and about 85 wt. % to about 99.1 wt. % of a solvent, and the suspension comprises from about 0.1 wt. % to about 50 wt. % of the core particles, from about 4 wt. % to about 10 wt. % of the enzyme immobilization material, and from about 50 wt. % to about 75 wt. % of the liquid medium. Other ways to make the casting solution include mixing the particles and the enzyme or organelle together in buffer to form a suspension and then adding solubilized immobilization material to complete the mixture or by combining all of the materials at once to form a suspension.

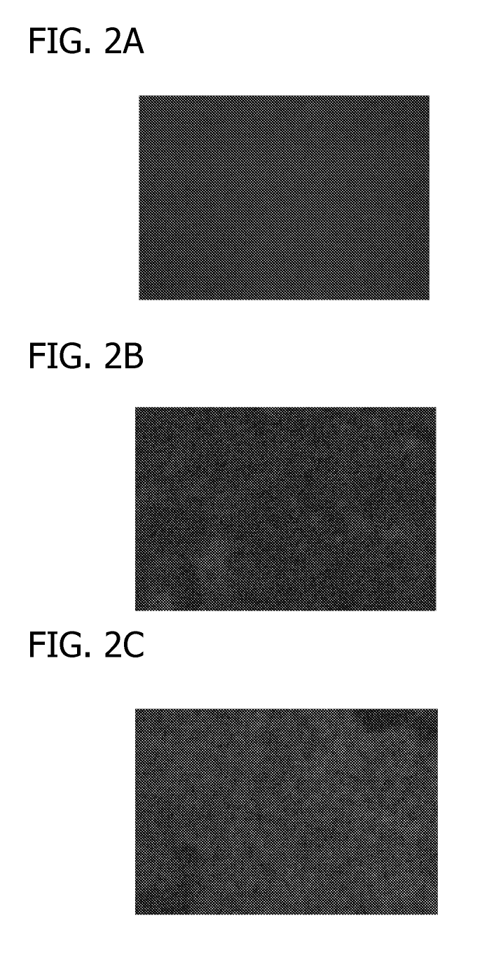

In various preferred embodiments, a mixture of enzyme, enzyme immobilization material, and optionally, electron mediators can be coated onto supporting particles using a spray coating/drying technique. For example, an airbrush (e.g., Paasche VL series) can be used to generate an aerosol of the components of the mixture and propel them towards a target. See FIG. 2.2. The aerosol is generated using compressed nitrogen gas regulated at about 25 psi. The mixture is airbrushed onto a surface such as a polycarbonate shield from a distance of about 40 cm from the tip of the airbrush to the shield. The airbrush can be moved in a raster pattern while moving vertically down the polycarbonate target in a zigzag pattern applying the casting solution. This procedure is used to minimize the coating thickness on the shield and minimize the particle-particle interaction while drying. The casting solution is allowed to dry on the shield for about 20 minutes before being collected by a large spatula/scraper.

### Substrate

Once the enzyme has been immobilized within the enzyme immobilization material, this immobilized enzyme can be deposited on a substrate. The substrate can be a material that provides the desired mechanical support necessary for the selected use. For example, the substrate may be a filter, a wire mesh, and the like when the immobilized enzyme is used as a catalyst for a chemical transformation. Further, the substrate can be an electron conducting material for a biosensor.

### Applications and Uses

Immobilized enzymes can be used as catalysts for a variety of chemical transformations. Enzymes can be immobilized in enzyme immobilization materials using processes described herein and deposited on various substrates. These immobilized enzymes can then contact reaction mixtures and catalyze the desired chemical transformation.

In particular, immobilized enzymes can be used for catalyzing a reaction wherein the enzyme immobilization material immobilizes and stabilizes the enzyme, is permeable to a compound smaller than the enzyme and is a micellar hydrophobically modified polysaccharide. In various preferred embodiments, the micellar hydrophobically modified polysaccharide is a hydrophobically modified chitosan or a hydrophobically modified alginate.

Further, immobilized enzymes can be used for detecting an analyte. The enzyme immobilization material immobilizes and stabilizes the enzyme, is permeable to a compound smaller than the enzyme and is a micellar hydrophobically modified polysaccharide. In various preferred embodiments, the micellar hydrophobically modified polysaccharide is a hydrophobically modified chitosan or a hydrophobically modified alginate.

For example, carbonic anhydrase is used to convert carbon dioxide to carbonic acid which then converts spontaneously to bicarbonate and carbonate. Following adjustment of the pH to favor carbonate formation, mineral ions are (e.g., Ca²⁺ or Mg²⁺) added to precipitate a carbonate salt that is easily stored. In this way, carbon dioxide is removed selectively from a gas and thus, it is sequestered. Alternatively, the carbonic acid formed from carbon dioxide is added to a carbonate slurry, forming bicarbonate that can be deposited in the ocean with little environmental impact. Carbon dioxide from a variety of sources, including fossil fuel combustion, calcining of limestone into lime, and various industrial effluents. Also, this conversion of carbon dioxide to carbonate and bicarbonate using carbonic anhydrase can be used to remove carbon dioxide from closed environments such as spaceships, space suits, planetary bases, and surface rovers. This type of reaction can also be used to maximize the growth of various photosynthetic organisms by sequestering carbon dioxide from the environment and using the sequestered carbon as the carbon source for growth of the organism. In one system, the organism is cyanobacteria that can synthesis hydrogen. Various uses of carbon sequestration systems using carbonic anhydrase are described in U.S. Pat. Nos. 6,348,347; 6,602,703; 6,908,507; and 7,132,090; these patents are herein incorporated by reference.

For preparation of various oligosaccharides, immobilized glycosyl hydrolases, such as chitinases, chitosanases, β-1,3-glucanases, β-fucosidases and glycosyl transferases can be immobilized. Depending on the enzyme used various mono- and di-saccharides can be produced. Further,

For example, enzymes are used to degrade stains in laundry soil, such as in detergents to break down and remove proteins from clothes. Generally, the enzymes used in detergents are proteases, amylases, carbohydrases, cellulases, and lipases. These enzymes can be immobilized and stabilized in enzyme immobilization materials described herein and dispersed in a detergent or in an aqueous carrier to provide a laundry soil treatment. The enzyme immobilization materials could stabilize the enzyme to the other components of the detergent and improve storage stability. Various detergent products containing enzymes are described in U.S. Pat. Nos. 7,179,780, 6,894,013, and 6,827,795; these patents are herein incorporated by reference.

Also, enzymes are used in wastewater treatment to break down various wastes in the stream. For example, lipases, cellulases, amylases, and proteases are used in addition to bacteria to eliminate various wastes. The components in specific waste streams and the appropriate enzymes used to degrade such waste streams are known to a person skilled in the art. For example, various enzymes used in wastewater treatment are disclosed in U.S. Pat. Nos. 7,053,130, 6,802,956, 5,531,898, and 4,882,059; these patents are herein incorporated by reference.

Another example of industrial use of enzymes is for converting corn or other cereals to high fructose corn syrup. Enzymes are used in three steps of high fructose corn syrup processing; these steps are liquefaction of the corn or cereal, saccharification of corn or other cereals to convert starch into sugars, and isomerization of glucose to fructose. In particular, glucose isomerase is used to convert glucose to fructose. The enzymes used could be advantageously immobilized in the enzyme immobilization materials described herein. This immobilization of the enzymes would be advantageous because the enzymes would be stabilized to denaturation from time, temperature, or less than optimum pH (particularly low pH). Further, the enzymes could be easily removed resulting in a more controlled process. Also, the immobilized enzymes could be easily separated from the high fructose corn syrup product. These processing steps are described in more detail in U.S. Pat. Nos. 5,593,868 and 4,567,142; these patents are herein incorporated by reference.

Additionally, enzymes are used in food processing because these processes require enzymes that catalyze various reactions of proteins. For example, in baking processes, fungal amylases, hemicellulase, pentosanases, xylanases, proteases, pullulanases, and acid proteinases are used for various purposes. In one process, fungal amylase is used to modify flours for baking and produce more uniform dough and products. Also, maltogenic amylases are used to extend shelf-life of various types of bread and pullulanase is used as an antistaling agent in baked goods. In brewing, amylases, proteases, β-glucanase, and acetolactate decarboxylase are used in various steps. For beer, some of these enzymes can be added to the malted barley to aid conversion of starch to fermentable sugars, to remove “chill haze” and to improve filtration. For cheese and whey production, proteases (e.g., pepsin) and lipases are used in the milk curdling and cheese ripening steps and lactase is used for production of whey syrup. Further, β-glucosidase transforms isoflavone phytoestrogens in soymilk. Each of these enzymes can be immobilized by the processes described herein and be advantageously used as catalysts for these reactions. These processes are described in more detail in U.S. Pat. Nos. 7,014,878, 6,936,289, 6,830,770, 4,358,462, and 6,372,268; these patents are herein incorporated by reference.

Various chemical synthesis processes use enzymes for esterifications, chiral synthesis, and interesterification and degumming of oils. For example, various carboxylic acid compounds including polymers having pendant carboxylic acid groups can be esterified with alcohols using various enzymes. The enzymes used to catalyze an esterification reaction are generally hydrolyic enzymes, specifically lipases, proteases, and esterases. Illustrative enzymes include Candida antarctica Lipase B (manufactured by Novozyme), Mucor meihei Lipase IM, Pseudomonas Cepacia Lipase PS-30, Pseudomonas aeruginosa Lipase PA, Pseudomonas fluoresenses Lipase PF, Aspergillus niger lipase, and Candida cylinderacea lipase from porcine pancreatic lipase. These reactions are described in more detail in U.S. Pat. Nos. 7,183,086 and 6,924,129; these patents are herein incorporated by reference.

For chemical synthesis, a variety of enzymes can catalyze various chemical transformations. For example, glutaminase is used to convert glutamine to glutamates, penicillin acylase is used in chemical synthesis, chloroperoxidase is used in steroid synthesis, aspartic β-decarboxylase is use to make L-alanine from L-aspartic acid, and cyclodextrin glycosyltransferase is used to make cyclodextrins from starch. Also, synthesis of various chiral compounds use enzymes. Subtilisin is used for the chiral resolution of chemical compounds or pharmaceuticals, aminoacylase can optically resolve amino acids, alcohol dehydrogenase is used in the chiral synthesis of chemicals and amino acid oxidase is used for resolution of racemic amino acid mixtures. Some of these reactions are described in more detail in U.S. Pat. Nos. 6,036,983, 5,358,860, 6,979,561, 5,981,267, 6,905,861, and 5,916,786; these patents are herein incorporated by reference.

Another reaction type that would greatly benefit from immobilized and stabilized enzymes are electrode systems using redox enzymes for enantioselective reductions and chemo-, regio-, and enantioselective oxidations. These reactions are especially useful when used at an electrode that is designed to provide enzyme-coupled cofactor regeneration. Such electrodes have been developed for NAD⁺- and NADP⁺-dependent enzymes. These reactions and electrodes are described in more detail in U.S. Pat. No. 6,991,926 and U.S. Patent Application Publication No. 2007/0231871; this patent and published application are herein incorporated by reference.

Oils can be interesterified or degummed using various enzymes. For example, enzymatic interesterification is an efficient way of controlling the melting characteristics of edible oils and fats. This is done by controlling the degree of conversion/reaction. No chemicals are used in the process and no trans fats are formed as in other production methods. An immobilized lipase can be used to interesterify fatty acids on oils and fats used in the production of margarine and shortening. Further, enzymatic processes can be used to remove gums in vegetable oil; this process is typically called degumming. Enzymatic degumming using a phospholipase enzyme converts non-hydratable lecithin (gums) to water-soluble lyso-lecithin, which is separated by centrifugation. Many of these processes are described in U.S. Pat. Nos. 6,162,623, 6,608,223, 7,189,544, and 6,001,640; these patents are herein incorporated by reference.

Further, immobilized lipases can be used to produce biodiesel. Various animal and plant fats contain triglycerides that can be transesterified with alcohols (e.g., methanol or ethanol) to form esters of the fatty acids of the triglycerides. These esters can be used as fuel. This transesterification process typically is very slow, but lipase catalysts can speed up this reaction to produce the biofuel without using harsh chemical agents.

Further, immobilized enzymes can be used in biosensors. Generally, these biosensors are used in diagnostic methods to sense various analytes in complex mixtures such as body fluids and industrial mixtures. For example, various sensors can be used to detect urea, uric acid, and cholesterol in various body fluids. Various sensors are described in U.S. Pat. No. 5,714,340; this patent is herein incorporated by reference. The immobilized enzymes or organelles of the present invention can be substituted for the enzymes used in biosensors to extend the useful life of the biosensor.

Immobilized enzymes can be used in various assay methods to determine the presence and/or concentration of various compounds or organisms in body fluids (e.g., saliva, blood, or urine). These assay methods use various enzymes to interact with an analyte and an enzyme inhibitor; these interactions allow for a large number of molecules to be formed or transformed from detection of one or a few analyte molecules or organisms. In turn, the enzyme activity is proportional to the analyte concentration and allows low concentrations of analyte to be detected. Analytes can range from small molecule pesticides and other toxins to microbes. Various assay probes having a high specificity for capture and detection of particular analytes can be used. Assay methods using enzyme amplification are described in U.S. Pat. Nos. 6,171,802, 6,383,763, and 4,067,774; these patents are herein incorporated by reference.

Also, immobilized enzymes can be used for purification of substances and separation of components in mixtures. For example, enzymes can be used to separate chiral molecules and isomers from racemic mixtures, to remove sulfur from oils, gases, and other industrial materials, to break down and separate lignocellulose components from plants, to treat and purify wastewater, and for food processing to remove undesirable compounds.

Further, immobilized enzymes can be used to catalyze various separations. During such a reaction, the enzyme can catalyze a transformation that aids in separating the components of a mixture. For example, carbonic anhydrase can be used to react with carbon dioxide in the air and convert it to carbonic acid that then can react to form carbonate salts. These carbonate salts can easily be isolated from other components of the air. Further, immobilized laccase enzymes can react with oxygen in the air to form peroxide and then water. The water is easily separated from the components of the air. For these extractions, an immobilized enzyme in a membrane can be used. Further, the extractions can be liquid/liquid or gas/liquid extractions.

Desulfurization of oils, gases, and other industrial materials use various enzymes. These enzymes are generally known as desulfurization enzymes. One important desulfurization enzyme is desulfinase. These desulfurization enzymes decompose thiophene in various petroleum products and remove sulfur atoms from the petroleum feed stocks prior to combustion. These processes are described in U.S. Pat. Nos. 6,461,859 and 7,045,314; these patents are herein incorporated by reference.

Lignin from various plants can be degraded by lignin peroxidase. Lignin peroxidase catalyzes a variety of oxidations to result in various cleavage and other oxidation products. These oxidation reactions include carbon-carbon bond cleavage of the propyl side chains of the lignin, hydroxylation of benzylic methylene groups, oxidation of benzyl alcohols to corresponding aldehydes or ketones, and phenol oxidation. These processes are described in more detail in U.S. Pat. No. 7,049,485; this patent is herein incorporated by reference.

Fruit juice is processed using pectinases, amylases, and cellulases that break down various structures of the fruit cells and enhance the juice extraction process. Polysaccharides released from cells during the fruit processing are insoluble in the juice and make the juice cloudy. To clarify the juice, pectinases and amylases are used to break down these insoluble polysaccharides to make soluble sugars. This process results in a clear juice that is sweetened due to the added soluble sugars. These processes are described in more detail in U.S. Pat. Nos. 5,585,128, 5,419,251, and 4,971,811; these patents are herein incorporated by reference.

Active surface films are coatings that include reactive substances and/or enzymes that can react with undesired contaminants to be self-cleaning or to remove various toxins from the surface. For example, active surface films can be included in food wraps, fibers, surface layers, bandages, or filters to break down toxins or kill bacteria. For example, enzymes that degrade various toxins could be included in fibers to destroy toxins used as biological weapons. Also, these same fibers could be coated with enzymes that kill or disrupt bacteria to prevent the wearer from contracting a bacterial infection.

Also, more than one enzyme or organelle can be immobilized to provide a multifunctional material that is able to catalyze more than one reaction. For example, a combination of the above described enzymes or organelles could be immobilized in one or more enzyme immobilization materials to provide a product that was able to catalyze more than one reaction.

Having described the invention in detail, it will be apparent that modifications and variations are possible without departing from the scope of the invention defined in the appended claims.

### EXAMPLES

### Example 1

### Immobilized Enzymes

Casting solutions for making the mixture-cast membranes of Nafion® and quaternary ammonium bromides were prepared as described below.

The following quaternary ammonium bromides were employed: ammonium bromide (Fisher), tetramethylammonium bromide (Aldrich), tetraethylammonium bromide (Fisher), tetrapropylammonium bromide (Aldrich), tetrabutylammonium bromide (Eastman), and tetrapentylammonium bromide (Aldrich).

Nafion® membranes incorporated with quaternary ammonium bromides were formed by co-casting the quaternary ammonium bromide with 5% by wt. Nafion® suspension (Solution Technologies, Inc.). The mixture-casting solutions were prepared by adding the quaternary ammonium bromides to the 5% by wt. suspension. All mixture-casting solutions were prepared so the concentration of quaternary ammonium bromides is in excess of the concentration of sulfonic acid sites in the Nafion® suspension. After optimization, it was determined that the most stable and reproducible membrane had a quaternary ammonium bromide concentration that is three times the concentration of the exchange sites.

One milliliter of the mixture-casting solution was placed in a weighing boat and allowed to dry. Previous studies have shown that all of the bromide ions that were introduced into a membrane were ejected from the membrane upon soaking that membrane in water. Therefore, 7.0 mL of 18 MΩ water were added to the weighing boats and allowed to soak overnight. The water was removed and the films were rinsed thoroughly with 18 MΩ water and dried. The salt-extracted films were then resuspended in 1.0 mL of lower aliphatic alcohols.

Enzyme/Nafion® casting solutions with an enzyme to TBAB ratio of 2:1 (usually 1200 μL of 1.0 μM enzyme: 600 μL) and 0.03 g NAD⁺ were vortexed in preparation for coating on electrode. The solution was pipetted onto the electrode, allowed to soak into the carbon-felt electrode and dried.

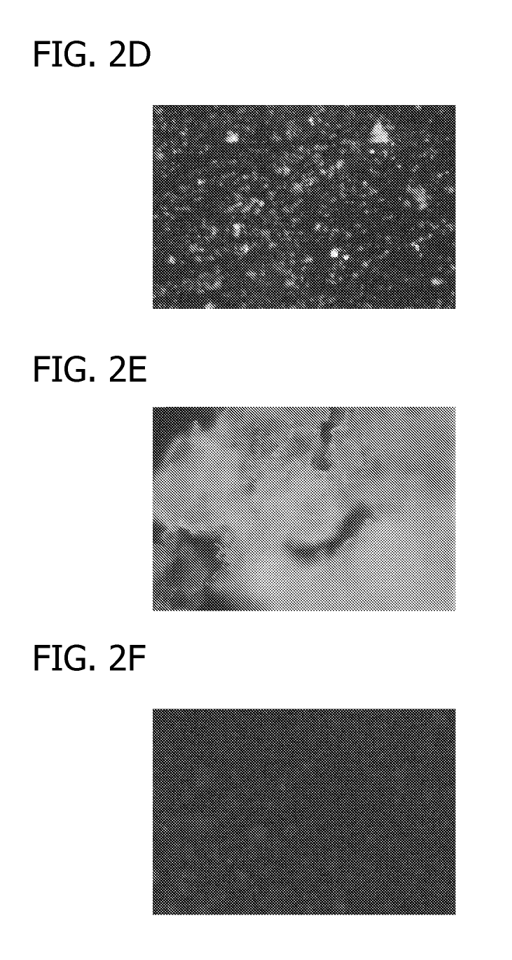

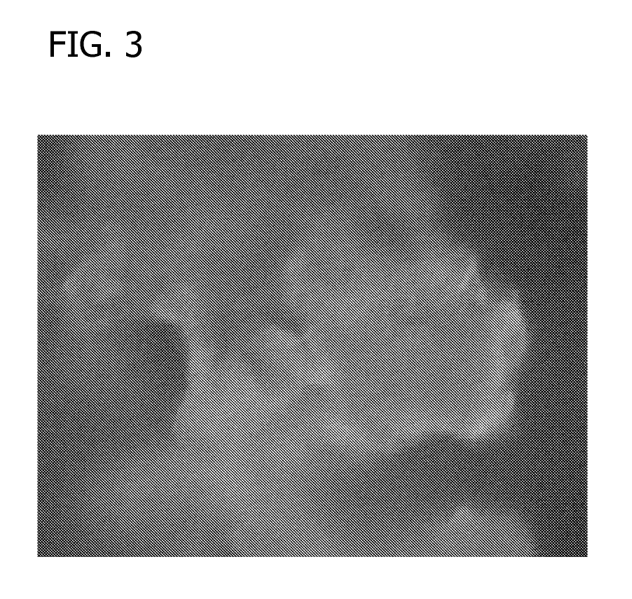

FIGS. 2-4 show fluorescence micrographs of enzymes immobilized in various modified perfluoro sulfonic acid-PTFE copolymer membranes that were treated with a NAD⁺ and fuel fluid solution in pH 7.15 phosphate buffer. The fluorescence micrographs that show fluorescence indicate that the enzyme immobilized in the particular modified perfluoro sulfonic acid-PTFE copolymer membrane are still catalytically active enzymes after immobilization. This is one way to determine whether the particular enzyme immobilization material will immobilize and stabilize the enzyme while retaining the enzyme's catalytic activity. The following Table 3 details the enzymes and membranes that were prepared and whether the enzymes retained catalytic activity after immobilization. Where the table indicates “yes,” the enzyme retained its catalytic activity after immobilization in the membrane and where the table indicates “no,” the enzyme did not retain its catalytic activity after immobilization in the membrane.

TABLE 3

| | Alcohol | Aldehyde | Formate | | | Formaldehyde |
|---|---|---|---|---|---|---|
| Membrane | DH | DH | DH | Glucose DH | Lactic DH | DH |
| Nafion ® | No | No | No | No | No | No |
| AmmoniumBr | No | No | No | No | No | No |
| TMABr | No | No | No | No | No | No |
| TEABr | No | No | No | No | No | No |
| TPropABr | Yes | No | No | No | Yes | No |
| TBABr | Yes | Yes | Yes | Yes | Yes | Yes |
| TPentABr | No | Yes | Yes | Yes | Yes | No |

### Example 2

### Enzyme Sensor

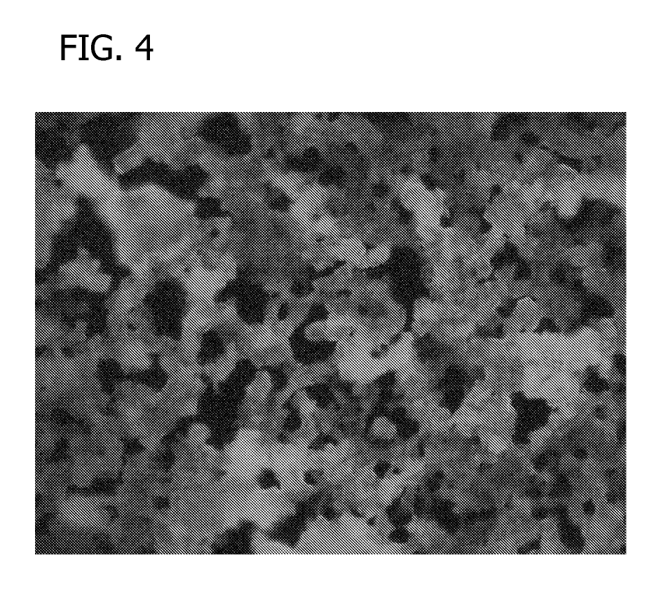

A glassy carbon electrode was modified with methylene green which was polymerized by chemical or electrochemical means. Alcohol dehydrogenase was immobilized in a tetrabutylammonium bromide-modified Nafion® membrane using the procedure described above in Example 1 and the immobilized enzyme was layered on the modified glassy carbon electrode and allowed to dry. After the modified glassy carbon electrode dried, it was placed in 1.5 mL of pH 7.15 phosphate buffer and allowed to equilibrate. After equilibration, amperometric sensing was performed at a potential of −0.4V. After a baseline reading was established, 1 mL of 8 mM ethanol solution in phosphate buffer was injected into the solution. FIG. 5 shows the graph of the current vs. time for the amperometric sensor.

### Example 3

### Preparation of Enzyme-Immobilized Salt-Extracted Membranes

### Preparation of Ru(bpy)₃⁺²/Nafion I

To prepare the Ru(bpy)₃⁺²/Nafion I Salt-Extracted Membrane (“Nafion I”), 0.15 millimols of Ru(bpy)₃ was added to 4 ml of Nafion®, mixed well for ˜3 to 4 hours by vortexing and using a sonicator in a constant temperature water bath. The mixture was then poured into a weighing boat to dry overnight. Once dry, the Ru(bpy)₃⁺²/Nafion® mixture was salt extracted by soaking into deionized water using vortex, followed by centrifugation. The extracted solution went from orange to clear when all salt was extracted. The salt-extracted membrane was rinsed and dried, then redissolved in 4 ml of 80% Ethanol (can be redissolved in a mixture of lower aliphatic alcohols containing up to 30% water).

The enzyme was immobilized in the Ru(bpy)₃⁺²/Nafion I Salt-Extracted Membrane by the following procedure. Approximately 1 mg of an electron mediator and approximately 0.5 to 1 mg of the cathode enzyme were added to 100 ml Ru(bpy)₃⁺²/Nafion III Salt-Extracted Membrane (supra) and mixed well (in this case, vortexed for 20 minutes).

### Preparation of Ru(bpy)₃⁺²/Nafion II:

To prepare Ru(bpy)₃⁺²/Nafion II Salt-Extracted Membrane (“Nafion II”), 0.3 millimoles of tetrabutylamonium bromide (TBAB) (0.09672 g) were added to each 1 ml of Nafion®, then mixed by vortex for 10 minutes. The mixture was then poured into a weighing boat to dry overnight. (At this point, the membrane mixture was light yellow.) Once dry, the TBAB treated Nafion® was soaked in deionized water for 24 h, then rinsed three (3) times with deionized water and allowed to dry. (At this point, the membrane mixture was clear.) The dry salt-extracted layer was then soaked over night in Ru(bpy)₃⁺² solution (1 mM Ru(bpy)₃⁺² dissolved in buffer, water or electrolyte), allowed to dry, then redissolved in 1 ml of ethanol. The enzyme was immobilized in the modified membrane according to the protocol set forth above.

### Preparation of Ru(bpy)₃⁺²/Nafion III

To prepare Ru(bpy)₃⁺²/Nafion III Salt-Extracted Membrane (“Nafion III”), 0.3 millimoles of tetrabutylamonium bromide (TBAB) (0.09672 g) were added to each 1 ml of Nafion®, then mixed by vortex for 10 minutes. The mixture was then poured into a weighing boat to dry overnight. (At this point, the membrane mixture was light yellow.) Once dry, the TBAB treated Nafion® was soaked in deionized water for 24 h, then rinsed three (3) times with deionized water and allowed to dry. (At this point, the membrane mixture was clear.) The dry salt-extracted layer was then redissolved in 1 ml of ethanol. The enzyme was immobilized in the modified membrane according to the protocol set forth above. The TBAB-modified Nafion® containing immobilized bilirubin oxidase was cast to an electrode, allowed to dry and then soaked in Ru(bpy)₃⁺² solution (supra) for up to 48 hours; preferably, for 2 to 3 hours before testing.

### Example 4

### Preparation of Alkyl Modified Chitosan

Medium molecular weight chitosan (available from Aldrich) (0.500 g) was dissolved by rapid stirring in 15 mL of 1% acetic acid. This resulted in a viscous gel-like solution and then 15 mL of methanol was added. The chitosan gel was allowed to stir for approximately 15 minutes, then 20 mL aldehyde (butanal, hexanal, octanal, or decanal) was added to the chitosan gel, followed by 1.25 g of sodium cyanoborohydride. The gel was continuously stirred until the suspension cooled to room temperature. The resulting product was separated by vacuum filtration and washed with 150 mL increments of methanol three times. The modified chitosan was then dried in a vacuum oven at 40° C. for two hours, leaving a flaky white solid. One percent by weight suspensions of each of the polymers were formed in 50% acetic acid, chloroform, and t-amyl alcohol.

### Example 5

### Preparation of Ru(bipyridine)₂(4-methyl-4′-(6-bromohexyl)-2,2′-bipyridine) Modified Chitosan

The preparation of Ru(bipyridine)₂(4-methyl-4′-(6-bromohexyl)-2,2′-bipyridine) modified chitosan started with the synthesis of a substituted bipyridine, 4-methyl-4′-(6-bromohexyl)-2,2′-bipyridine. To prepare the substituted bipyridine, 50 mL THF containing 1.69 g 4,4′-dimethyl-2,2′-bipyridine was added dropwise over 30 minutes to 4.1 mL of THF containing 9.1 mmol lithium diisopropylamine. This mixture was stirred for 1.5 hours, then cooled to 0° C., followed by dropwise addition of 9.2 mmol dibromoalkane of desired chain length with stirring. This mixture was stirred for 1.5 hours, quenched with ice water, and extracted with ether. The residue was recrystallized 3 times from ethyl acetate. Once the 4-methyl-4′-(6-bromohexyl)-2,2′-bipyridine was prepared, it was reacted to form the Ru(bipyridine)₂(4-methyl-4′-(6-bromohexyl)-2,2′-bipyridine) by refluxing 1.315 g of Ru(bpy)₂Cl₂(in its hydrate form), 0.8201 g of 4-methyl-4′-(6-bromohexyl)-2,2′-bipyridine, and 0.76 g sodium bicarbonate in 60 mL of 2:3 methanol-water solution until the Ru(bpy)₂Cl₂ was depleted. The depletion of Ru(bpy)₂Cl₂ was determined by UV-Vis absorption data. The resulting complex was precipitated by adding 4 mL of 3 M ammonium hexafluorophosphate (or a sodium or potassium perchlorate salt), followed by recrystallization from acetone/CH₂Cl₂. This reaction sequence yielded 77% Ru(bipyridine)₂(4-methyl-4′-(6-bromohexyl)-2,2′-bipyridine)⁺².

After its preparation, 137 mg Ru(bipyridine)₂(4-methyl-4′-(6-bromohexyl)-2,2′-bipyridine)⁺² was dissolved in a mixture of 5 mg of deacetylated chitosan in 1% acetic acid and DMF (1:1, 1 mL). This mixture was heated at 90° C. for 12 hours. After the reaction period, acetonitrile was added to precipitate Ru(bipyridine)₂(4-methyl-4′-(6-hexyl)-2,2′-bipyridine)⁺² modified chitosan. The precipitate was collected and purified by dissolution in 1% acetic acid, then recrystallized in methanol and dried under reduced pressure.

### Example 6

### Fluorescence Imaging of Hydrophobically Modified Chitosans

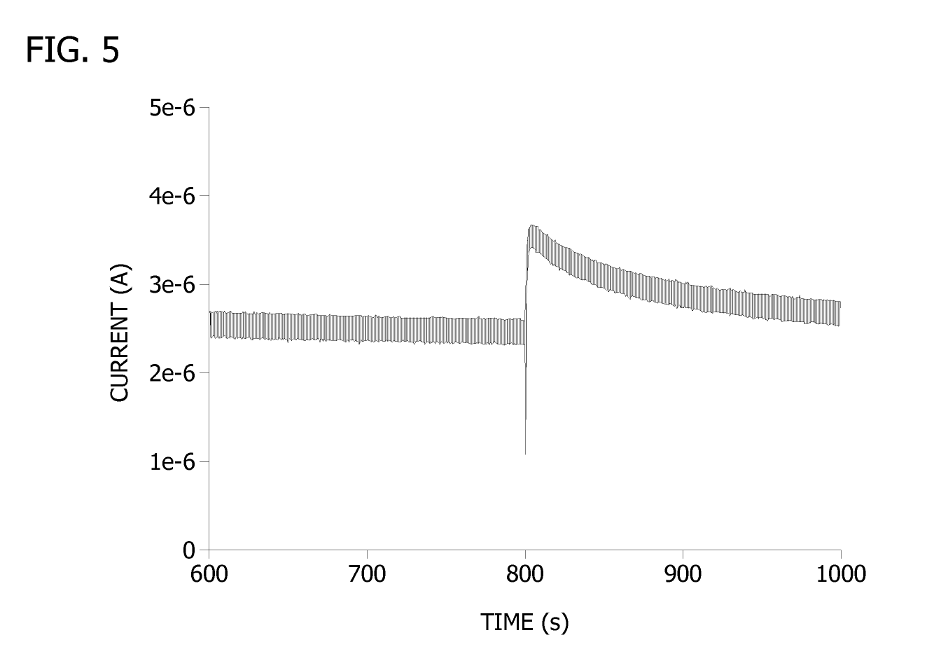

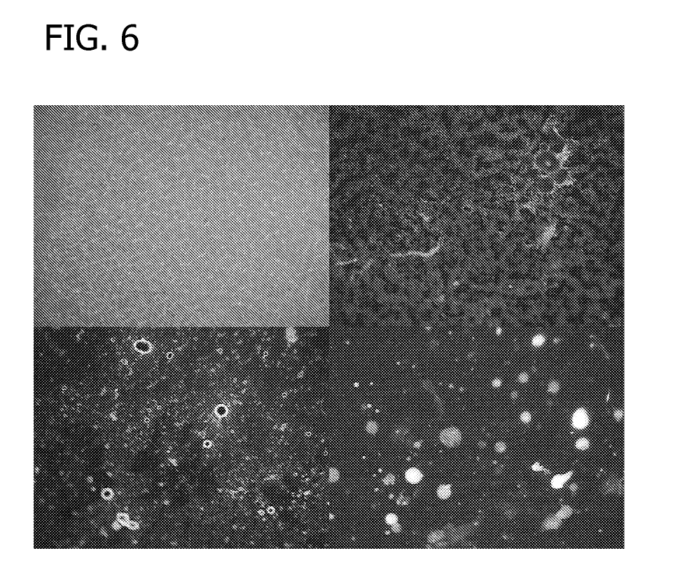

Each polymer suspension (2 μL) was cast onto a glass microscope slide (Fisher) and dried in the desiccator. A 20 μL volume of 0.01 mM Ru(bpy)₃²⁺ or 0.01 mM FITC was pipetted onto the polymer cast and allowed to soak for two minutes. After soaking, the slides were rinsed with 18 MΩ water and allowed to dry in the desiccator. The polymers were imaged using an Olympus BX60M epifluorescence microscope (Melville, N.Y.). The polymers were observed under a 40× ultra-long working distance lens with a video camera (Sony SSC-DC50A). Fluorescence excitation was achieved with a mercury lamp. A frame grabber card (Integral Technologies, Inc., Indianapolis, Ind.) was used to acquire images, and the images were analyzed using SPOT software (Diagnostic Instruments, Inc.) on a Dell PC. Fluorescence imaging of each of the hydrophobically modified polyelectrolytes in Ru(bpy)₃⁺² and fluorescein was performed to determine the morphological effects of the hydrophobic modification. FIG. 6 is representative fluorescence micrographs of hydrophobically modified chitosan in Ru(bpy)₃⁺². It can be seen that aggregates form within the hydrophobically modified chitosans and that the morphology changes with alkyl chain length. The butyl modified chitosan appears to have small, fibrous interconnects, whereas the hexyl modified chitosan has large domains containing smaller micellar domains. As the alkyl chain length increases, the number of micellar domains decreases, but the size of the domain increases. Fluorescence micrographs of unmodified chitosan do not show distinct domains, so micellar structure was not observed for unmodified chitosan. FIG. 7 is representative fluorescence micrographs of hydrophobically modified chitosan membranes soaked in FITC. The same morphological changes can be observed with either the cationic or the anionic fluorescent dye.

### Example 7

### Electrochemical Measurements of Hydrophobically Modified Chitosans

Glassy carbon working electrodes (3 mm in diameter, CH Instruments) were polished on a Buehler polishing cloth with 0.05 micron alumina and rinsed in 18 MΩ water. Each polymer suspension (2 μL) was cast onto a glassy carbon electrode surface and allowed to dry in a vacuum desiccator until use. Cyclic voltammetry was used to measure the flux of the redox species through the polymer membrane at the electrode surface. The working electrodes were allowed to equilibrate in a 1.0 mM redox species solution containing 0.1 M sodium sulfate as the supporting electrolyte along with a platinum mesh counter electrode and measured against a saturated calomel reference electrode. The redox species studied were caffeine, potassium ferricyanide, and Ru(bpy)₃²⁺. The data were collected and analyzed on a Dell computer interfaced to a CH Instruments potentiostat model 810. Cyclic voltammetry was performed at scan rates ranging from 0.05 V/s to 0.20 V/s. All experiments were performed in triplicate and reported uncertainties correspond to one standard deviation.

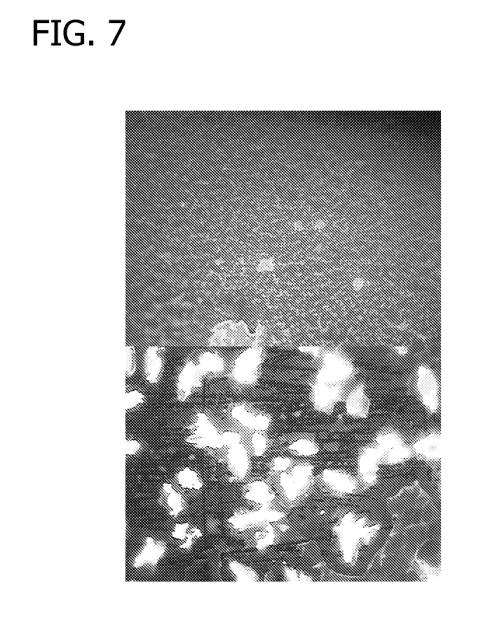

Cyclic voltammetric studies of the two hydrophobically modified polyelectrolytes were conducted as a function of the alkyl chain length of the hydrophobic modification. All cyclic voltammetric experiments showed linear iₚ vs v^{1/2} plots, signifying transport-limited electrochemistry. Since electrochemical flux is a function of concentration as shown in Equation 2, KD^{1/2} values are reported in this paper as a concentration independent method of comparing fluxes.

$$Flux = \frac{i}{nFA} = \frac{2.69 \times {10}^{5} {n}^{3 / 2} {AC}^{*} {v}^{1 / 2} {KD}^{1 / 2}}{nFA} \qquad Equation 2$$

where i is the peak current, n is the number of electrons transferred, F is Faraday's constant, A is the area of the electrode, C* is the concentration of redox species, v is the scan rate, K is the extraction coefficient, and D is the diffusion coefficient. FIG. 8 shows the KD^{1/2} values for flux of caffeine through hydrophobically modified chitosan as a function of the alkyl chain length of the modifier and the solvent in which the polymer is resuspended. The solvent determines the degree of swelling of the polymer during re-casting. Most literature studies on chitosan and chitosan derivatives employ acetic acid as the solvent for resuspension, however, it is important to note from the KD^{1/2} values for chloroform provides a higher average flux. Unmodified chitosan is only soluble in the acetic acid solution. The KD^{1/2} value for unmodified chitosan in caffeine is 5.52 (±0.14)×10⁻³. It is clear that hydrophobic modification of chitosan can decrease the flux of caffeine, but cannot make appreciable increases in flux.

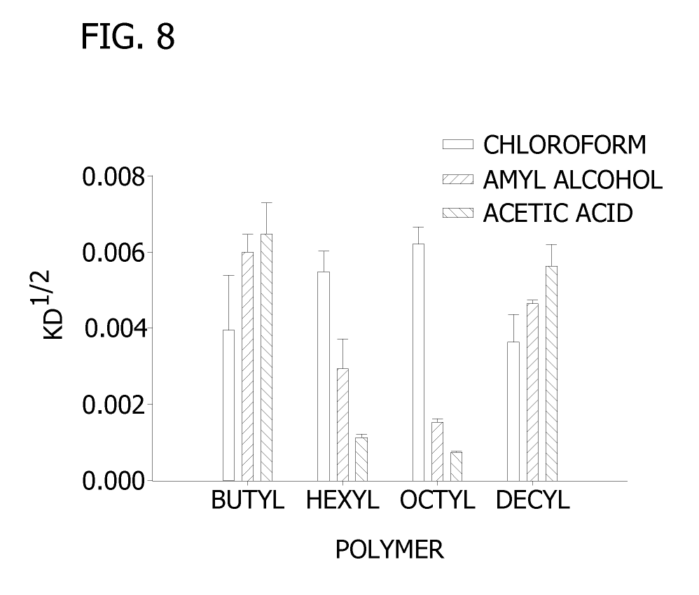

On the other hand, transport of large, hydrophobic ions, like Ru(bpy)₃⁺², can be greatly affected by small changes in pore structure/size. FIG. 9 shows KD^{1/2} values for transport of Ru(bpy)₃⁺² through hydrophobically modified chitosan membranes. The KD^{1/2} value for Ru(bpy)₃⁺² transport through unmodified chitosan is 2.17 (±0.33)×10⁻⁴. It is evident that hydrophobic modification of chitosan increases the transport of Ru(bpy)₃⁺² in all cases, by as much as 11.1 fold for octyl modified chitosan membrane resuspended in t-amyl alcohol.

### Example 8

### Preparation of Electrodes

A solution of 2 wt. % of a hydrophobically modified chitosan polymer was suspended in t-amyl alcohol and a solution of glucose oxidase was added. This solution was pipetted onto an electrode material. This electrode material was typically a carbon cloth, or other carbon material.

### Example 9

### Glucose Oxidase Activity Tests for Hydrophobically Modified Chitosans

Glucose oxidase (GOx) catalyzes the oxidation of β-D-glucose to D-glucono-δ-lactone with the concurrent release of hydrogen peroxide. It is highly specific for β-D-glucose and does not act on α-D-glucose. In the presence of peroxidase, hydrogen peroxide enters into a second reaction in the assay involving p-hydroxybenzoic acid and 4-amino antipyrine with the quantitative formation of quinoneimine dye complex, which is measured at 510 nm. The activity of GOx enzyme was measured in each of the hydrophobically modified Nafion® and chitosan membranes. The absorbance was measured at 510 nm against water after immobilizing the GOx enzyme within the hydrophobically modified chitosan membranes, and casting it in a plastic vial. All experiments were performed in triplicate and reported uncertainties correspond to one standard deviation.

As described above and tabulated in Table 2, the highest enzyme activity was observed for glucose oxidase in a hexyl modified chitosan suspended in t-amyl alcohol. These immobilization membranes showed a 2.53 fold increase in GOx enzyme activity over enzyme in buffer.

### Example 10

### Chitosan-butyl Electrodes

Glucose dehydrogenase. Electrodes were made from 1 cm² AvCarb™ carbon paper. The electrodes were electropolymerized in 0.4 mM methylene green, 0.1 M sodium nitrate and 10 mM sodium borate by performing cyclic voltammetry from −0.3 V to 1.3 V for 12 sweep segments at a scan rate of 0.05 V/s. They were then rinsed and allowed to completely dry in a vacuum dessicator. Chitosan mixtures were prepared by mixing 0.01 g hydrophobically modified chitosan (butyl, hexyl, octyl or decyl) with 1 mL Nafion® DE 520 and vortexing with mixing beads for 1 hour. A 40 μL aliquot of the chitosan/Nafion® mixture was then mixed with a 20 μL aliquot of glucose dehydrogenase (1 mg enzyme in 10 mL pH 7.15 phosphate buffer) for 1 minute. The chitosan/enzyme mixture was pipetted onto the anode and allowed to completely dry in the vacuum dessicator.

Alcohol dehydrogenase. The electrode containing alcohol dehydrogenase was prepared by the same procedure as described for glucose dehydrogenase above except alcohol dehydrogenase was substituted for glucose dehydrogenase.

### Example 11

### Chitosan-butyl Electrodes

Bilirubin Oxidase. Chitosan mixtures were prepared by mixing 0.01 g hydrophobically modified chitosan (butyl, hexyl, octyl or decyl) with 1 mL Nafion® DE 520 and vortexing with mixing beads for 1 hour. A 40 μL aliquot of the chitosan/Nafion® mixture was then mixed with a 20 μL aliquot of bilirubin oxidase (1 mg enzyme in 10 mL pH 7.15 phosphate buffer) for 1 minute. The chitosan/enzyme mixture was pipetted onto a 1 cm² piece of carbon paper to fabricate the electrode and it was allowed to completely dry in the vacuum dessicator.

### Example 12

### Preparation of Alkyl Modified Alginate

Alginate membranes incorporated with quaternary ammonium bromides were formed by co-casting the quaternary ammonium bromide with 3 wt. % alginate suspension. The polymer used was either ultra low, low, or medium molecular weight alginate. The mixture-casting solutions were prepared by adding the quaternary ammonium bromides to the 3 wt. % suspension. All mixture-casting solutions were prepared so the concentration of quaternary ammonium bromides is in excess of the concentration of carboxylic acid sites in the alginate suspension. After optimization, it was determined that the most stable and reproducible membrane has a quaternary ammonium bromide concentration that is three times the concentration of the exchange sites.

One milliliter of the casting solution was placed in a weighing boat and allowed to dry. 7.0 mL of 18 MΩ water were added to the weighing boats and allowed to soak overnight. The water was removed and the films were rinsed thoroughly with 18 MΩ water and dried. Then, the films were resuspended in 1.0 mL of methanol. Ammonium bromide salts of tetrapropylammonium (T3A), tetrapentylammonium (T5A), tetrahexylammonium (T6A), tetraheptylammonium (T7A), trimethylicosylammonium (TMICA), trimethyloctyldecylammonium (TMODA), trimethylhexyldecylammonium (TMHDA), trimethyltetradecylammonium (TMTDA), trimethyloctylammonium (TMOA), trimethyldodecylammonium (TMDDA), trimethyldecylammonium (TMDA), trimethylhexylammonium (TMHA) tetrabutylammonium (TBA), triethylhexylammonium (TEHA) were used as alginate modifiers to see which yielded the best micellar structure. The micellar structure is important for effective immobilization of an enzyme.

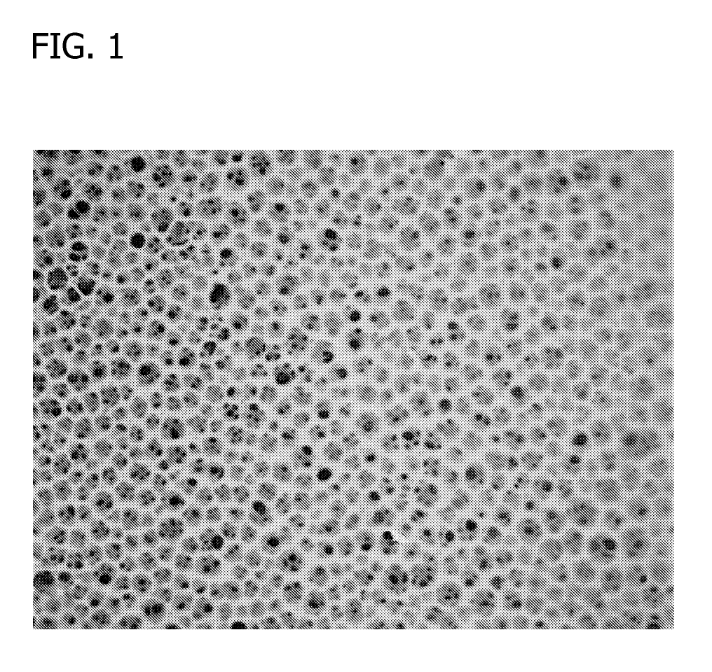

To determine the pore characteristics, three drops of each polymer were then placed on a slide and left to dry. After completely drying, they were soaked in 1 mM Ru(bpy)⁺² in ethanol for at least 3 hours. After being rinsed off with ethanol, the polymers were left to dry before being imaged with a fluorescence microscope to see the micellar structure. An example of the structure is shown in FIG. 1.

In another experiment, ultralow molecular weight alginate and dodecylamine were placed in 25% ethanol and refluxed to produce a dodecyl-modified alginate by amidation of the carboxylic acid groups.

### Example 13

### Preparation of Alginate Electrodes

A solution of 3 wt. % of an alginate polymer modified with a hydrophobic ammonium cation described in Example 12 is suspended in t-amyl alcohol and a solution of enzyme (e.g., alcohol dehydrogenase, glucose dehydrogenase, bilirubin oxidase, glucose oxidase) is added. This solution is pipetted onto an electrode material. This electrode material is typically a carbon cloth, or other carbon material.

### Example 14

### CuPCTSA-TBAB Coated poly(styrene-co-divinylbenzene)

In this example poly(styrene-co-divinylbenzene) particles were coated with a mixture of tetrabutylammonium bromide (TBAB)-modified Nafion® immobilization material and a water soluble dye that acts as an electron mediator for enzymatic reduction of oxygen to water. In order to prepare these particle, a dye solution of 0.080 g copper (II) phthalocyanine tetrasulphonic acid (CuPCTSA) in 4.00 mL TBAB modified Nafion® (5 wt %) in ethanol was combined with a particle suspension of 2.00 g poly(styrene-co-divinylbenzene) particles in 4.00 mL 0.5 M phosphate buffer, pH 7.2. The dye solution was added to the particle suspension and vortex mixed for several seconds until a substantially uniform mixture was achieved. The entire mixture was then airbrushed onto a polycarbonate shield. The mixture was allowed to dry on the shield for 20 minutes before being collected and stored dry in a scintillation vial.

The resulting coated particles were blue in color and retention of the dye was confirmed by packing the coated particles in a column and passing several hundred mL of water across them. No detectable levels of dye were found in the mobile phase eluting from the column. The thickness of the coating layer was designed to be 0.07 micron thick and the average diameter of the particles before coating was 8 microns. The surface area of the particles (pre-coated) was calculated to be 0.75 m/g². The resulting loading of the electron mediator was 4 wt %.

### Example 15

### Alcohol Dehydrogenase-TBATFB Coated Doped Polypyrrole/Carbon Black Composite

Doped polypyrrole/carbon black composite (Sigma, catalog number 530573) particles were coated with a mixture of tetrabutylammonium tetrafluoroborate (TBATFB)-modified Nafion® immobilization material and alcohol dehydrogenase enzyme. A solution of 0.080 g freeze dried alcohol dehydrogenase (ML57) and 2.00 mL 0.5 M phosphate buffer (pH 7.2) was prepared. A suspension of 4.00 mL TBATFB modified Nafion (5 wt %) in ethanol, 1.00 g doped polypyrrole/carbon black composite, and 4.00 mL 0.5 M phosphate buffer (pH 7.2) was prepared. The enzyme solution and the suspension were then vortex mixed for several seconds until a substantially uniform mixture was formed. The entire mixture was then airbrushed onto a polycarbonate shield in a process similar to Example 14. The resulting product was stored dry in a scintillation vial at 4° C.

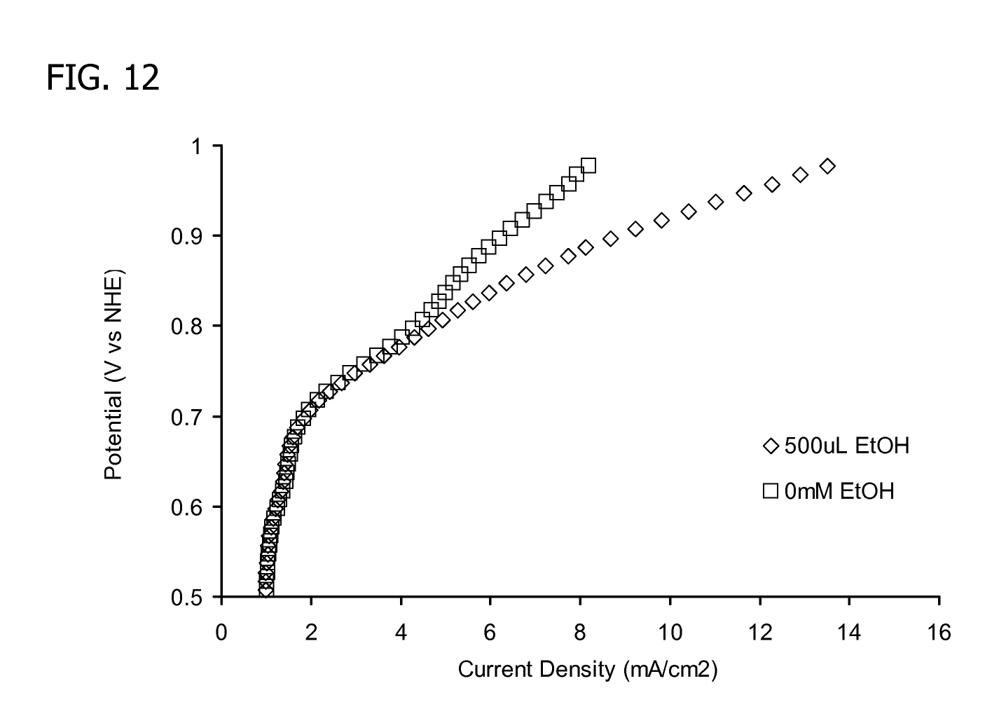

The resulting coated particles were black in color and retention of the enzyme activity was confirmed using standard spectrophotometric assay and electrochemical evaluation. The evaluation was made versus a normal hydrogen reference electrode (NHE). See FIG. 12. The resulting loading of the enzyme was 6.25 wt %.

### Example 16

### Alcohol Dehydrogenase-Ru(II)(NH₃)₆-TBATFB Coated Doped Polypyrrole/Carbon Black Composite

Doped polypyrrole/carbon black composite particles (Sigma, catalog number 530573) were coated with a mixture of TBATFB-modified Nafion®, alcohol dehydrogenase, and Ru(II)(NH₃)₆. Ru(II)(NH₃)₆ is an electron mediator for enzymatic oxidation of ethanol. A solution containing 0.080 g freeze dried alcohol dehydrogenase (ML59), 0.100 g Ru(II)(NH₃)₆, and 4.00 mL 0.5 M phosphate buffer (pH 7.2) was prepared. A suspension containing 6.00 mL 0.5 M phosphate buffer (pH 7.2), 2.00 mL TBATFB modified Nafion (5 wt %) in ethanol, and 2.00 g doped polypyrrole/carbon black composite was also prepared. The solution and suspension were combined and vortex mixed for several seconds to form a substantially uniform mixture. The entire mixture was then airbrushed onto a polycarbonate shield in a process similar to Example 1. The resulting product was stored dry in a scintillation vial at 4° C.

The resulting coated particles were black in color and retention of the enzyme activity was confirmed using standard spectrophotometric assay. The coating thickness was not calculated due to the unknown surface area of the particles. The resulting loading of the enzyme and electron mediator was 10.3 and 12.8 wt %, respectively.

### Example 17

### Starch-Consuming Amylase Enzyme Activity

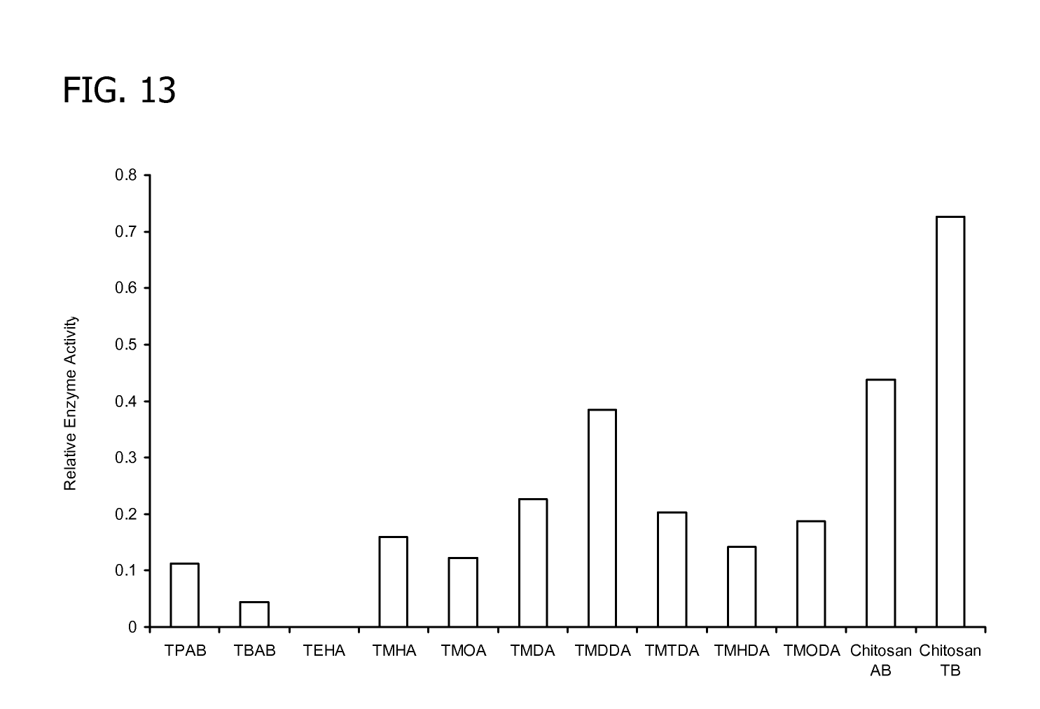

The enzymatic assay used for amyloglucosidase (EC 3.2.1.3) is published by Sigma and available at http://www.sigmaaldrich.com/img/assets/18200/Amyloglucosidasel.pdf and is based on the literature procedure described in Bergmeyer, H. U., Gawehn K., and Grassl, M. (1974) Methods of Enzymatic Analysis (Bergmeyer, H. U. ed.) Second Edition, Volume 1, 434-435. This procedure was modified slightly by immobilizing the amyloglucosidase with the desired enzyme immobilization material at the bottom of a cuvette rather than adding the amyloglucosidase to the solution. Enzyme immobilization materials tested were Nafion® modified with tetrapropropylammonium bromide (TPAB), tetrabutylammonium bromide (TBAB), triethylhexylammonium bromide (TEHA), trimethylhexylammonium (TMHA), trimethyloctylammonium (TMOA), trimethyldecylammonium (TMDA), trimethyldodecylammonium (TMDDA), trimethyltetradecylammonium (TMTDA), trimethylhexyldecylammonium (TMHDA), trimethyloctyldecylammonium (TMODA), butyl modified chitosan suspended in acetate buffer (Chitosan AB), and butyl modified chitosan suspended in t-amyl alcohol (Chitosan TB). The enzyme immobilization material that provided the greatest relative activity for the starch consuming amylase was butyl modified chitosan suspended in t-amyl alcohol (Chitosan TB). See FIG. 13.

### Example 18

### Maltose Consuming Amylase Enzyme Activity

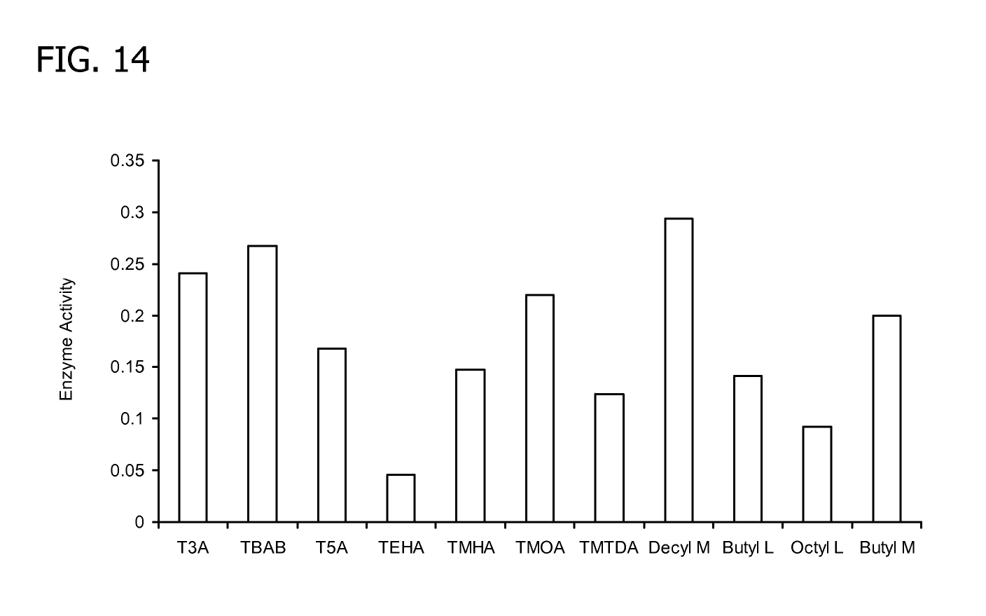

The procedure described in Example 17 was used to determine the activity of maltose consuming amylase immobilized in various enzyme immobilization materials. The published procedure was modified as described in Example 17 and further modified by substituting maltose for starch. Enzyme immobilization materials tested were Nafion® modified with tetrapropropylammonium bromide (T3A), tetrabutylammonium bromide (TBAB), tetrapentylammonium bromide (T5A), triethylhexylammonium bromide (TEHA), trimethylhexylammonium (TMHA), trimethyloctylammonium (TMOA), trimethyltetradecylammonium (TMTDA), medium molecular weight decyl modified chitosan (Decyl M), low molecular weight butyl modified chitosan (Butyl L), low molecular weight octyl modified chitosan (Octyl L), and medium molecular weight butyl modified chitosan (Butyl M). The enzyme immobilization material that provided the greatest relative activity for the maltose consuming amylase was medium molecular weight decyl modified chitosan (Decyl M). See FIG. 14.

### Example 19

### Temperature and pH Tolerance of Immobilized Lipase

Procedure for Tetrabutylammonium Bromide (TBAB) Modified Nafion Immobilized Lipase Enzyme Coated on Poly(styrene-co-divinyl benzene) beads. Lipase enzyme (5 mg) was dissolved in 5 mL of 50 mM Tris-HCl buffer (pH 7.2) solution. After preparing this 1 mg/mL lipase enzyme solution, it was transferred into a 20 mL capacity polypropylene sample vial. Eight 3 mm diameter zirconia beads were added to the propylene vial to help the agitation during the vortexing steps (8 zirconia beads were satisfactory to provide a good agitation for total volume of up to 20 mL liquid). Tetrabutylammonium bromide (TBAB) modified Nafion (3.0 mL; 5 wt % in ethanol) was transferred into the vial and this enzyme/polymer suspension was vortexed with zirconia beads for two minutes. After this first addition, another 3.0 mL of TBAB modified Nafion solution (5% in Ethanol) was added and vortexed for two minutes. A total of 6 mL TBAB modified Nafion solution was added in small portions to prevent the alcohol shocking of the enzyme. At this point, the enzyme was immobilized and was ready to be placed on a support material. Poly(styrene-co-divinyl benzene) (PS-co-DVB) beads (8 μm mean particle size, Sigma Cat. #: 468312-100G) (1.00 g) were mixed with the immobilized enzyme solution and vortexed with zirconia beads for three minutes to evenly coat the PS-co-DVB beads. After coating the beads with the immobilized enzyme, the mixture was spray dried onto a glass sheet at room temperature and the glass sheet was left to dry open to ambient air.

Procedure for Tetraethylammonium Bromide (TEAB) Modified Nafion Immobilized Lipase Enzyme Coated on Poly(styrene-co-divinyl benzene) beads. Lipase enzyme (25 mg) was dissolved in 5 mL of 50 mM Tris-HCl buffer (pH 7.2) solution. After preparing this 5 mg/mL lipase enzyme solution, it was transferred into a 20 mL capacity polypropylene sample vial. Eight 3 mm diameter zirconia beads are added into the propylene vial to help the agitation during the vortexing steps (8 zirconia beads are satisfactory in number to provide a good agitation for total volume of up to 20 mL liquid). Tetraethylammonium bromide (TEAB) modified Nafion (2.5 mL; 5 wt % in ethanol) was transferred into the vial and this enzyme/polymer suspension was vortexed with zirconia beads for two minutes. After this first addition, another 2.5 mL of TEAB modified Nafion solution (5% in Ethanol) was added and vortexed for two minutes. Total of 5 mL TEAB modified Nafion solution was added in small portions to prevent the alcohol shocking of the enzyme, which may even possibly denature the enzyme due to extreme local environment changes. At this point, the enzyme was already immobilized and ready to be placed on a support material. Poly(styrene-co-divinyl benzene) (PS-co-DVB) (1.00 g) beads (8 μm mean particle size, Sigma Cat. #: 468312-100G) mixed with immobilized enzyme solution and vortexed with zirconia beads for three minutes to evenly coat the PS-co-DVB beads. Once beads were coated with the immobilized enzyme, it was spray dried onto a glass sheet at room temperature. Once it was sprayed, the glass sheet was left to dry open to ambient air.

Procedure for Hexanal Modified Chitosan Immobilized Lipase Enzyme Coated on Poly(styrene-co-divinyl benzene) beads. Lipase enzyme (25 mg) is dissolved in 5 mL of 50 mM Tris-HCl buffer (pH 7.2) solution. After preparing this 5 mg/mL lipase enzyme solution, it was transferred into a 20 mL capacity polypropylene sample vial. Eight 3 mm diameter zirconia beads are added into the propylene vial to help the agitation during the vortexing steps (8 zirconia beads are satisfactory in number to provide a good agitation for total volume of up to 20 mL liquid). Hexanal modified chitosan (2.5 mL; 1 wt % in proprietary solvent mixture) was transferred into the vial and this enzyme/polymer suspension was vortexed with zirconia beads for two minutes. After this first addition, another 2.5 mL of hexanal modified chitosan (1 wt % in proprietary solvent mixture) was added and vortexed for two minutes. Total of 5 mL of hexanal modified chitosan (1 wt % in proprietary solvent mixture) solution was added in small portions to prevent the solvent shocking of the enzyme, which may even possibly denature the enzyme due to extreme local environment changes. At this point, the enzyme was already encapsulated and ready to be placed on a support material. Poly(styrene-co-divinyl benzene) (PS-co-DVB) beads (8 μm mean particle size, Sigma Cat. #: 468312-100G) (1.00 g) mixed with immobilized enzyme solution and was vortexed with zirconia beads for three minutes to evenly coat the PS-co-DVB beads. Once beads were coated with the immobilized enzyme, it was spray dried onto a glass sheet at room temperature. Once it was sprayed, the glass sheet was left to dry open to ambient air.

Lipase Immobilization Activity Assay Protocol. Temperature tolerance of Candida antarctica lipase B (CAL-B) was investigated. The free lipase (Novozyme's CAL-B free in solution) along with various experimental immobilized lipases (TBAB/Nafion, TEAB/Nafion, and/or Hexanal modified chitosan) were tested. As a comparison, Novozyme's/Codexis's version of immobilized enzyme labeled Novozym® 435 (hereinafter Novo 435). All lipase samples were weighed out to contain 5 mg/ml of lipase in each sample. All lipase activity assays were conducted at pH=7.2 (50 mM Tris-HCl) [1.4 mL] in the presence of 12 mM p-nitrophenylbutyrate dissolved in 100% dimethylsulfoxide (DMSO) [0.1 mL], 10% sodium deoxycholate (to clear up turbidity) [0.4 mL] and either free lipase, Novozym® 435 immobilized lipase, or an experimental immobilized lipase [0.1 mL] for a total of 2.0 mL of reaction volume. The absorbance change due to enzyme activity was measured at 405 nm in a spectrophotometer over a span of 3 to 5 minutes. All enzyme activities were performed at room temperature. Samples exhibiting enzyme activity showed increased absorbance changes from the 0 min. time point and the reaction color went from colorless to bright yellow.

Temperature tolerance. Enzyme samples were incubated at room temperature, 65° C., and 95° C. over a span of 2 to 64 hours as indicated in the tables below. Typically, the enzyme samples were incubated for 2-4 hours and 18-24 hours and the enzyme activities were measured after the incubation period.

pH tolerance. Enzyme samples were incubated at room temperature and pH=12.5 (50 mM NaOH) or pH=1.1 (50 mM HCl).

TABLE 4
Enzyme samples incubated in 50 mM HCl (pH 1.1)
at room temperature for 1 hour.

| | Sample | | |
|---|---|---|---|
| Time (min) | Free Lipase | Novo 435 | TEAB/Lipase |
| 0 | 0.001 | 0.061 | 0.272 |
| 1 | 0.028 | 0.158 | 0.739 |
| 2 | 0.064 | 0.261 | 1.3 |
| 3 | 0.104 | 0.351 | 1.761 |
| Δ Abs. 405 nm | 0.103 | 0.29 | 1.489 |

TABLE 5
Enzyme samples incubated in 50 mM HCl (pH 1.1)
at room temperature for 1 hour.

| | Sample | | |
|---|---|---|---|
| Time (min) | Free Lipase | Novo 435 | TEAB/Lipase |
| 0 | 0.056 | 0.009 | 0.33 |
| 1 | 0.07 | 0.03 | 0.959 |
| 2 | 0.087 | 0.05 | 1.668 |
| 3 | 0.103 | 0.083 | 2.125 |
| Δ Abs. 405 nm | 0.047 | 0.074 | 1.795 |

TABLE 6
Enzyme samples incubated in 50 mM NaOH (pH 12.5)
at room temperature for 2 hours.

| | Sample | | |
|---|---|---|---|
| Time (min) | Free Lipase | Novo 435 | Chitosan/Lipase |
| 0 | 0.012 | 0 | 0.137 |
| 1 | 0.018 | 0.017 | 0.184 |
| 2 | 0.03 | 0.037 | 0.255 |
| 3 | 0.045 | 0.066 | 0.345 |
| Δ Abs. 405 nm | 0.033 | 0.066 | 0.208 |

TABLE 7
Enzyme samples incubated in 50 mM HCl (pH 1.1)
at room temperature for 2 hours.

| | Sample | | |
|---|---|---|---|
| Time (min) | Free Lipase | Novo 435 | Chitosan/Lipase |
| 0 | 0.073 | 0.006 | 0.335 |
| 1 | 0.946 | 0.018 | 0.355 |
| 2 | 1.768 | 0.033 | 0.366 |
| 3 | 2.161 | 0.051 | 0.391 |
| Δ Abs. | 2.088 | 0.045 | 0.056 |
| 405 nm | | | |

TABLE 8
Enzyme samples incubated in 50 mM NaOH
(pH 12.5) at room temperature for 4 hours.

| | Sample | | |
|---|---|---|---|
| Time (min) | Free Lipase | Novo 435 | TEAB/Lipase |
| 0 | 0.025 | 0.044 | 0.362 |
| 1 | 0.027 | 0.058 | 0.991 |
| 2 | 0.04 | 0.081 | 1.635 |
| 3 | 0.054 | 0.124 | 2.043 |
| Δ Abs. | 0.029 | 0.08 | 1.681 |
| 405 nm | | | |

TABLE 9
Enzyme samples incubated in 50 mM HCl (pH 1.1)
at room temperature for 4 hours.

| | Sample | | |
|---|---|---|---|
| Time (min) | Free Lipase | Novo 435 | TEAB/Lipase |
| 0 | 0.004 | 0 | 0.182 |
| 1 | 0.023 | 0.023 | 0.387 |
| 2 | 0.053 | 0.042 | 0.731 |
| 3 | 0.083 | 0.069 | 1.096 |
| Δ Abs. 405 nm | 0.079 | 0.069 | 0.914 |

TABLE 10
Enzyme samples incubated in 50 mM NaOH (pH 12.5)
at room temperature for 20 hours.

| | Sample | | |
|---|---|---|---|
| Time (min) | Free Lipase | Novo 435 | TEAB/Lipase |
| 0 | 0 | 0.031 | 0.363 |
| 1 | 0.007 | 0.039 | 0.766 |
| 2 | 0.019 | 0.058 | 1.298 |
| 3 | 0.031 | 0.178 | 1.76 |
| Δ Abs. 405 nm | 0.031 | 0.147 | 1.397 |

TABLE 11
Enzyme samples incubated in 50 mM HCl (pH 1.1)
at room temperature for 20 hours.

| | Sample | | |
|---|---|---|---|
| Time (min) | Free Lipase | Novo 435 | TEAB/Lipase |
| 0 | 0 | 0.024 | 0.217 |
| 1 | 0.012 | 0.277 | 0.401 |
| 2 | 0.039 | 0.561 | 0.709 |
| 3 | 0.061 | 0.84 | 1.093 |
| Δ Abs. 405 nm | 0.061 | 0.816 | 0.876 |

TABLE 12
Enzyme samples incubated in 50 mM NaOH (pH 12.5)
at room temperature for 24 hours.

| | Sample | | |
|---|---|---|---|
| Time (min) | Free Lipase | Novo 435 | Chitosan/Lipase |
| 0 | 0 | 0.013 | 0.162 |
| 1 | 0.012 | 0.025 | 0.195 |
| 2 | 0.025 | 0.042 | 0.255 |
| 3 | 0.042 | 0.06 | 0.303 |
| Δ Abs. | 0.042 | 0.047 | 0.141 |
| 405 nm | | | |

TABLE 13
Enzyme samples incubated in 50 mM HCl (pH 1.1)
at room temperature for 24 hours.

| | Sample | | |
|---|---|---|---|
| Time (min) | Free Lipase | Novo 435 | Chitosan/Lipase |
| 0 | 0.085 | 0.033 | 0.457 |
| 1 | 0.832 | 0.045 | 0.467 |
| 2 | 1.539 | 0.063 | 0.487 |
| 3 | 2.012 | 0.081 | 0.513 |
| Δ Abs. | 1.927 | 0.048 | 0.056 |
| 405 nm | | | |

TABLE 14
Enzyme samples incubated at room temperature for 2 hours.

| | Sample | | |
|---|---|---|---|
| Time (min) | Free Lipase | Novo 435 | Chitosan/Lipase |
| 0 | 0 | 0.093 | 0.165 |
| 1 | 2.24 | 0.688 | 1.002 |
| 2 | finished | 1.257 | 1.706 |
| 3 | finished | 1.74 | 2.135 |
| Δ Abs. | 2.24 | 1.647 | 1.97 |
| 405 nm | | | |

TABLE 15
Enzyme samples incubated at 65° C. for 2 hours.

| | Sample | | |
|---|---|---|---|
| Time (min) | Free Lipase | Novo 435 | Chitosan/Lipase |
| 0 | 0.06 | 0 | 0.229 |
| 1 | 0.2 | 0.028 | 1.07 |
| 2 | 0.362 | 0.058 | 1.776 |
| 3 | 0.529 | 0.087 | 2.166 |
| Δ Abs. | 0.469 | 0.087 | 1.937 |
| 405 nm | | | |

TABLE 16
Enzyme samples incubated at 95° C. for 2 hours.

| | Sample | | |
|---|---|---|---|
| Time (min) | Free Lipase | Novo 435 | Chitosan/Lipase |
| 0 | 0.04 | 0.006 | 0.195 |
| 1 | 0.052 | 0.013 | 0.197 |
| 2 | 0.075 | 0.019 | 0.231 |
| 3 | 0.101 | 0.026 | 0.276 |
| Δ Abs. | 0.061 | 0.02 | 0.081 |
| 405 nm | | | |

TABLE 17
Enzyme samples incubated at room temperature for 24 hours.

| | Sample | | |
|---|---|---|---|
| Time (min) | Free Lipase | Novo 435 | Chitosan/Lipase |
| 0 | 0.09 | 0.313 | 0.252 |
| 1 | 1.826 | 0.907 | 1.371 |
| 2 | 2.234 | 1.454 | 2.122 |
| 3 | finished | 1.846 | finished |
| Δ Abs. | 2.144 | 1.533 | 1.87 |
| 405 nm | | | |

TABLE 18
Enzyme samples incubated at 65° C. for 24 hours.

| | Sample | | |
|---|---|---|---|
| Time (min) | Free Lipase | Novo 435 | Chitosan/Lipase |
| 0 | 0 | 0.004 | 0.23 |
| 1 | 0.023 | 0.069 | 0.599 |
| 2 | 0.051 | 0.135 | 1.065 |
| 3 | 0.08 | 0.206 | 1.533 |
| Δ Abs. | 0.08 | 0.202 | 1.303 |
| 405 nm | | | |

TABLE 19
Enzyme samples incubated at 95° C. for 24 hours.

| | Sample | | |
|---|---|---|---|
| Time (min) | Free Lipase | Novo 435 | Chitosan/Lipase |
| 0 | 0.003 | 0.025 | 0.24 |
| 1 | 0.005 | 0.025 | 0.24 |
| 2 | 0.01 | 0.026 | 0.248 |
| 3 | 0.019 | 0.032 | 0.254 |
| ΔAbs at | 0.016 | 0.007 | 0.014 |
| 405 nm | | | |

TABLE 20
Enzyme samples incubated at the indicated temperature for 18 hours.

| | Sample | | |
|---|---|---|---|
| | Free Lipase | Free Lipase | Free Lipase |
| Time (min) | at RT | at 65° C. | at 95° C. |
| 0 | 0.232 | 0.001 | 0.001 |
| 1 | 1.643 | −0.001 | 0.004 |
| 2 | 2.265 | 0.004 | 0.005 |
| 3 | 2.317 | 0.008 | 0.006 |
| 4 | 2.317 | 0.007 | 0.006 |
| 5 | 2.317 | 0.004 | −0.008 |
| ΔAbs at | 2.085 | 0.003 | −0.009 |
| 405 nm | | | |
| ΔAbs at | 2.085 | 0.007 | 0.005 |
| 405 nm for | | | |
| 0-3 min | | | |

| | Sample | | |
|---|---|---|---|
| | Immobilized 435 | Immobilized 435 | Immobilized 435 |
| Time (min) | Lipase at RT | Lipase at 65° C. | Lipase at 95° C. |
| 0 | 0.001 | −0.026 | −0.001 |
| 1 | 0.267 | 0.004 | 0.001 |
| 2 | 0.514 | 0.04 | 0.01 |
| 3 | 0.77 | 0.078 | 0.005 |
| 4 | 1.021 | 0.114 | 0.01 |
| 5 | 2.12 | 0.209 | 0.02 |
| ΔAbs @ | 2.119 | 0.235 | 0.021 |
| 405 nm | | | |
| ΔAbs at | 0.769 | 0.104 | 0.006 |
| 405 nm for | | | |
| 0-3 min | | | |

| | Sample | | |
|---|---|---|---|
| | TEAB/Lipase | | TEAB/Lipase |
| Time (min) | at RT | TEAB/Lipase at 65° C. | at 95° C. |
| 0 | 0.244 | 0.252 | 0.4 |
| 1 | 0.93 | 0.451 | 0.431 |
| 2 | 1.782 | 0.7 | 0.469 |
| 3 | 2.223 | 1.015 | 0.518 |
| 4 | 2.298 | 1.395 | 0.558 |
| 5 | 2.308 | 1.726 | 0.594 |
| ΔAbs @ | 2.064 | 1.474 | 0.194 |
| 405 nm | | | |
| ΔAbs at | 1.986 | 0.763 | 0.118 |
| 405 nm for | | | |
| 0-3 min | | | |

TABLE 21
Enzyme samples incubated at the indicated temperature for 64 hours.

| | Sample | | |
|---|---|---|---|
| Time | TEAB/Lipase | TEAB/Lipase | TEAB/Lipase |
| (min) | at RT | at 65° C. | at 95° C. |
| 0 | 0.3 | 0.269 | 0.25 |
| 1 | 0.61 | 0.34 | 0.25 |
| 2 | 1.087 | 0.44 | 0.25 |
| 3 | 1.604 | 0.563 | 0.25 |
| 4 | 2.037 | 0.715 | 0.25 |
| 5 | 2.287 | 0.849 | 0.25 |
| ΔAbs at | 1.987 | 0.58 | 0 |
| 405 nm | | | |

Sample

| Time | | Immobilized 435 |
|---|---|---|
| (min) | Free Lipase at RT | Lipase at 65° C. |
| 0 | 0.24 | 0.006 |
| 1 | 1.74 | 0.015 |
| 2 | 2.345 | 0.022 |
| 3 | 2.377 | 0.033 |
| 4 | N/A | 0.051 |
| 5 | N/A | 0.093 |
| ΔAbs at | 2.137 | 0.087 |
| 405 nm | | |

The data can be converted into enzyme activity units by using the following equation Enzyme Units=((ΔAbsorbance at 405 nm/# min)×0.002 L)/(1 cm×0.0178 μmol⁻¹ cm⁻¹) wherein the molar absorptivity of p-nitrophenol is 1.78×10⁴ M⁻¹ cm⁻¹ and the μmolar absorptivity of p-nitrophenol is 0.0178 μmol⁻¹ cm⁻¹.

TABLE 22
Summary for various experiments wherein enzyme activity is in Enzyme Units (EU)

| Enzyme | RT for 18 h | 65° C. for 18 h | 95° C. for 18 h | % RT at 65 | % RT at 95 |
|---|---|---|---|---|---|
| free lipase | 0.078089888 | 0.000262172 | 0.000187266 | 0.33573141 | 0.2398082 |
| Novo 435 | 0.028801498 | 0.003895131 | 0.000224719 | 4.98800959 | 0.2877698 |
| TEAB/lipase | 0.074382022 | 0.028576779 | 0.004419476 | 36.5947242 | 5.6594724 |
| Enzyme | RT for 24 h | 65° C. for 24 h | 95° C. for 24 h | % RT at 65 | % RT at 95 |
| free lipase | 0.080299625 | 0.002996255 | 0.000599251 | 3.73134328 | 0.7462687 |
| Novo 435 | 0.05741573 | 0.007565543 | 0.000262172 | 9.42164179 | 0.3264925 |
| chitosan/lipase | 0.070037453 | 0.048801498 | 0.000524345 | 60.7742537 | 0.6529851 |

| | pH = 12.5 for | % of optimal |
|---|---|---|
| Enzyme | 20 h | at 12.5 |
| free lipase | 0.001161049 | 1.486810552 |
| Novo 435 | 0.005505618 | 7.050359712 |
| TEAB/lipase | 0.052322097 | 67.00239808 |
| | pH = 12.5 for | % of optimal |
| Enzyme | 24 h | at 12.5 |
| free lipase | 0.001573034 | 1.958955224 |
| Novo 435 | 0.0017603 | 2.192164179 |
| chitosan/lipase | 0.005280899 | 6.576492537 |

For table 22, the enzyme activity in EU was determined for the first three rows using the ΔAbs at 405 nm for 0-3 minutes rather than the ΔAbs at 405 nm for 0-5 minutes. The data columns labeled “% RT at 65” or “% RT at 95” were calculated by the following equation:

(EU for the enzyme sample)/(EU for the free lipase at room temperature for the same amount of time)×100%.

The data columns labeled “% optimal at 12.5” were calculated by the following equation:

(EU for the enzyme sample)/(EU for the free lipase at room temperature for the similar amount of time)×100%.

### Example 20

### pH Tolerance of Immobilized Laccase

DS Elat Electrode Painted with PVP-TBAB and BP460 Filler (pH 1.78). Poly (4-vinylpyridine) (PVP) (29.4 mg), 410 μL of 19.6% tetrabutylammonium bromide (TBAB) modified Nafion® (in EtOH), 360 mg of Black Pearls carbon filler, and Nafion® ionomer (1.4 mL of 5% solution) was mixed and sonicated in a vial and this slurry was painted on a DS Elat carbon electrode. The electrode was left open to room air to dry at room temperature for 40 minutes. A 1 cm² piece of electrode was cut and used for half cell testing. This 1 cm² electrode was partially submerged in 0.2 M phosphate buffer (pH 1.78) solution during the half cell testing.

DS Elat Electrode PVP-TBAB and BP460 Filler and Free Enzyme in Buffer Solution (pH 1.78). Poly (4-vinylpyridine) (PVP) (29.4 mg), 410 μL of 19.6% tetrabutylammonium bromide (TBAB) modified Nafion® (in EtOH), 360 mg of Black Pearls carbon filler, and 1.4 mL of 5% Nafion® ionomer was mixed and sonicated in a vial and this slurry was painted on DS Elat carbon electrode. The electrode was left open to room air to dry at room temperature for 40 minutes. A 1 cm² piece of electrode was cut and used for half cell testing. This 1 cm² electrode was partially submerged in 0.5 mg Laccase enzyme containing 0.2 M phosphate buffer (pH 1.78) solution during the half cell testing.

DS Elat Electrode Painted with Sigma Lac immobilized with PVP-TBAB encapsulant and BP460 Filler Carbon (pH 1.78). Laccase (120 mg) immobilized with poly(4-vinylpyridine)/TBAB, 360 mg of Black Pearls carbon filler, and 1.8 mL of 5% Nafion® ionomer was mixed and sonicated in a vial and this slurry was painted on DS Elat carbon electrode. The electrode was left open to room air to dry at room temperature for 40 minutes. A 1 cm² piece of electrode was cut and used for half cell testing. This 1 cm² electrode was partially submerged in 0.2 M phosphate buffer (pH 1.78) solution during the half cell testing.

DS Elat Electrode PVP-TBAB and BP460 Filler and Free Enzyme in Buffer Solution (Optimum pH; pH 5). Poly (4-vinylpyridine) (PVP) (29.4 mg), 410 μL of 19.6% tetrabutylammonium bromide (TBAB) modified Nafion® (in EtOH), 360 mg of Black Pearls carbon filler, and 1.4 mL of 5% Nafion® ionomer was mixed and sonicated in a vial and this slurry was painted on DS Elat carbon electrode. The electrode was left open to room air to dry at room temperature for 40 minutes. A 1 cm² piece of electrode was cut and used for half cell testing. This 1 cm² electrode was partially submerged in 0.5 mg Laccase enzyme containing 1 M phosphate buffer (pH 5) solution during the half cell testing.

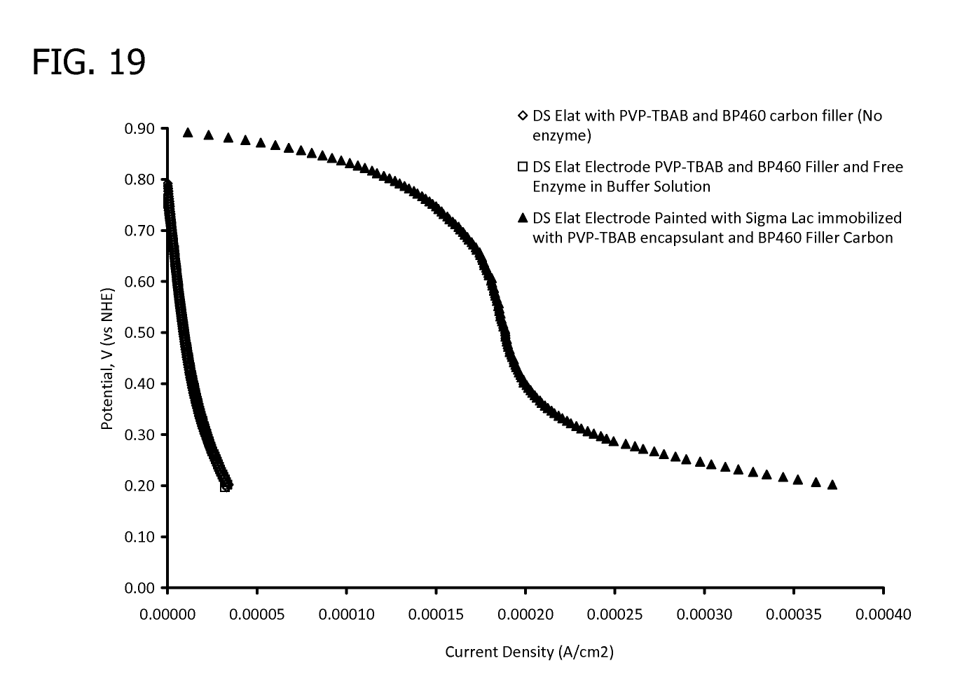

A polarization curve was obtained using a CH Instruments Potentiostat. The half cell testing was done with a platinum mesh counter electrode and a Ag/AgCl reference electrode. A graph of the potential versus the current density using a half cell having the laccase electrode at the cathode is shown in FIG. 19 wherein all potentials in the figure are versus NHE. This graph compares the immobilized laccase operating at pH 1.78 to otherwise identical electrodes containing no laccase, free laccase operating at pH 1.78, or free laccase operating at its optimum pH of 5. The immobilized laccase has much greater activity (as shown by the current density) than the free laccase in this system.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

Whilst the description and claims of this application detail various alternatives for each feature of the invention, it will be understood that each of these features can be combined in any of the possible combinations and thus each alternative for each feature may be combined with any or all alternatives from some or all of the other features and thus each combination is therefore disclosed herein.

1-57. (canceled)

58. An enzyme immobilized in a micellar or inverted micellar immobilization material capable of immobilizing and stabilizing the enzyme, the material being permeable to a compound smaller than the enzyme and either:
(a) when exposed to a temperature of at least about 65° C. for at least about 18 hours, the stabilized enzyme retains at least about 12% of an activity of an otherwise identical free enzyme exposed to room temperature for the same amount of time;
(b) when exposed to a temperature of at least about 95° C. for at least about 18 hours, the stabilized enzyme retains at least about 1% of an activity of an otherwise identical free enzyme exposed to room temperature for the same amount of time;
(c) when exposed to a pH of greater than about 12.5 for at least about 18 hours, the stabilized enzyme retains at least about 9% of an activity of an otherwise identical free enzyme exposed to its optimal pH for the same amount of time; or
(d) when exposed to a pH of less than about 2, the stabilized enzyme retains at least about 10% of its initial bioelectrocatalytic activity for at least about 1 hour when continuously catalyzing a chemical transformation.

59. The immobilized enzyme of claim 58 wherein when exposed to a temperature of at least about 65° C. for at least about 18 hours, the stabilized enzyme retains at least about 12% of an activity of an otherwise identical free enzyme exposed to room temperature for the same amount of time.

60. The immobilized enzyme of claim 58 wherein when exposed to a temperature of at least about 95° C. for at least about 18 hours, the stabilized enzyme retains at least about 1% of an activity of an otherwise identical free enzyme exposed to room temperature for the same amount of time.

61. The immobilized enzyme of claim 58 wherein when exposed to a pH of greater than about 12.5 for at least about 18 hours, the stabilized enzyme retains at least about 9% of an activity of an otherwise identical free enzyme exposed to its optimal pH for the same amount of time.

62. The immobilized enzyme of claim 58 wherein when exposed to a pH of less than about 2, the stabilized enzyme retains at least about 10% of its initial bioelectrocatalytic activity for at least about 1 hour when continuously catalyzing a chemical transformation.

63. The immobilized enzyme of claim 59 wherein the stabilized enzyme retains at least about 25% of the activity of an otherwise identical free enzyme exposed to room temperature for the same amount of time.

64. The immobilized enzyme of claim 60 wherein the stabilized enzyme retains at least about 5% of the activity of an otherwise identical free enzyme exposed to room temperature for the same amount of time.

65. The immobilized enzyme of claim 61 wherein the stabilized enzyme retains at least about 25% of an activity of an otherwise identical free enzyme exposed to its optimal pH for the same amount of time.

66. The immobilized enzyme of claim 62 wherein the stabilized enzyme retains at least about 30% of its initial bioelectrocatalytic activity for at least about 48 hours.

67. The immobilized enzyme of claim 58 wherein the enzyme comprises a lipase, a glucose isomerase, a nitrilase, a glucose oxidase, a protease, a carbonic anhydrase, a pepsin, an amylase, a fungal amylase, a maltogenic amylase, a cellulase, a lactase, an esterase, a carbohydrase, a hemicellulase, a pentosanase, a xylanase, a pullulanase, a β-glucanase, an acetolactate decarboxylase, a β-glucosidase, a glutaminase, a penicillin acylase, a chloroperoxidase, an aspartic β-decarboxylase, a cyclodextrin glycosyltransferase, a subtilisin, an aminoacylase, an alcohol dehydrogenase, an amino acid oxidase, a phospholipase, a urease, a cholesterase, a desulfinase, a lignin peroxidase, a pectinase, an oxidoreductase, a dextranase, a glucosidase, a galactosidase, a glucoamylase, a maltase, a sucrase, an invertase, a naringanase, a bromelain, a ficin, a papain, a pepsin, a peptidase, a chymosin, a thermolysin, a trypsin, a triglyceridase, a pregastric esterase, a phosphatase, a phytase, an amidase, a glutaminase, a lysozyme, a catalase, a dehydrogenase, a peroxidase, a lyase, a fumarase, a histadase, an aminotransferase, a ligase, a cyclase, a racemase, a mutase, an oxidase, a reductase, a ligninase, a laccase, a chloroperoxidase, a haloperoxidase, a hydrogenase, a nitrogenase, an oxynitrilase, or a combination thereof.

68. The immobilized enzyme of claim 58 wherein the enzyme comprises a lipase.

69. The immobilized enzyme of claim 58 wherein the immobilization material comprises a cation modified perfluoro sulfonic acid-PTFE copolymer or cation-modified alginate modified with a hydrophobic cation larger than NH₄⁺.

70. The immobilized enzyme of claim 69 wherein the hydrophobic cation comprises tetraethylammonium, tetrapropylammonium (T3A), tetrapentylammonium (T5A), tetrahexylammonium (T6A), tetraheptylammonium (T7A), trimethylicosylammonium (TMICA), trimethyloctyldecylammonium (TMODA), trimethylhexyldecylammonium (TMHDA), trimethyltetradecylammonium (TMTDA), trimethyloctylammonium (TMOA), trimethyldodecylammonium (TMDDA), trimethyldecylammonium (TMDA), trimethylhexylammonium (TMHA), tetrabutylammonium (TBA), triethylhexylammonium (TEHA), and combinations thereof.

71. A particle comprising a core coated with an immobilized enzyme of claim 58.

72. The particle of claim 71 wherein the enzyme is entrapped within the immobilization material.

73. A process for catalyzing a reaction comprising catalyzing the reaction with an immobilized enzyme wherein the enzyme is immobilized in an enzyme immobilization material that immobilizes and stabilizes the enzyme, the material being permeable to a compound smaller than the enzyme, and wherein the reaction catalyzed is selected from
(a) conversion of carbon dioxide to carbonic acid;
(b) enantioselective oxidation or reduction of organic substrates with optional cofactor regeneration;
(c) reactive separation of a component or components from a mixture;
(d) esterification of a carboxylic acid with an alcohol;
(e) liquefaction of corn or other cereals;
(f) saccharification of corn or other cereals to convert starch into sugars;
(g) isomerization of glucose to fructose;
(h) synthesis of chiral compounds;
(i) interesterification of oils;
(j) degumming oil;
(k) treating wastewater (reaction);
(l) clarifying fruit juice;
(m) producing glucose by the starch process;
(n) producing glucose and galactose from lactose;
(o) synthesizing compounds having peptide bonds;
(p) producing 6-aminopenicillic acid from penicillin G;
(q) converting sugars to alcohol;
(r) removing sulfur from petroleum fractions;
(s) converting acrylonitrile to acrylamide;
(t) converting 3-cyanopyridine to nicotinamide; and
(u) degrading stains in a laundry soil.

74. The process of claim 73 wherein the reactive separation of a component or components from a mixture comprises separating or removing the substance from the mixture using an immobilized enzyme wherein the enzyme is entrapped in a micellar or inverted micellar enzyme immobilization material.

75. The process of claim 74 wherein the mixture is a racemic mixture, petroleum product, wastewater, biomass, or food.

76. The process of claim 73 wherein the reaction catalyzed is conversion of carbon dioxide to carbonic acid.

77. The process of claim 73 wherein the enzyme is entrapped in a micellar or inverted micellar immobilization material.
